(19) United States
(12) Patent Application Publication
(10) Pub. No.: US 2020/0123539 A1
(43) Pub. Date: Apr. 23, 2020

(54) METHOD AND SYSTEM FOR LIBRARY PREPARATION WITH UNIQUE MOLECULAR IDENTIFIERS

(71) Applicant: uBiome, Inc., San Francisco, CA (US)

(72) Inventors: Zachary APTE, San Francisco, CA (US); Jessica RICHMAN, San Francisco, CA (US); Daniel AL-MONACID, San Francisco, CA (US); Juan JIMINEZ, San Francisco, CA (US); Rodrigo ORTIZ, San Francisco, CA (US); Eduardo MORALES, San Francisco, CA (US); Paulo COVARRUBIAS, San Francisco, CA (US); Eduardo OLIVARES, San Francisco, CA (US); Nicolas ORDENES, San Francisco, CA (US); Luis LEON, San Franscisco, CA (US)

(21) Appl. No.: 16/624,816

(22) PCT Filed: Jun. 20, 2018
(86) PCT No.: PCT/US2018/038628

§ 371 Date: Dec. 19, 2019

Related U.S. Application Data
(60) Provisional application No. 62/522,293, filed on Jun. 20, 2017.
(60) Provisional application No. 62/582,162, filed on Nov. 6, 2017.

(51) Int. Cl. C12N 15/10

(52) U.S. Cl. CPC C12N 15/1093; C12N 15/1065

(57) ABSTRACT

Embodiments of a method 100 and/or system 200 or library preparation for sequencing associated with microorganisms can include: preparing a set of unique molecular identifier (UMI)-based molecules associated with one or more targets; preparing a set of sequencing-based primers; generating a set of tagged target molecules based on the set of UMI-based molecules and one or more samples associated with the one or more targets; and/or generating a set of sequencing-ready tagged target molecules based on the tagged target molecules and the set of sequencing-based primers.

### CROSS-REFERENCE TO RELATED APPLICATIONS

This application claims the benefit of U.S. Provisional Application Ser. No. 62/522,293 filed 20 Jun. 2017, and U.S. Provisional Application Ser. No. 62/582,162 filed 6 Nov. 2017, which are each incorporated in its entirety herein by this reference.

### TECHNICAL FIELD

The disclosure generally relates to genomics and molecular biology.

### BACKGROUND

Next Generation Sequencing (NGS) technologies (e.g., NGS platforms) can reduce the cost of DNA and/or other nucleic sequencing, improve the quality of information obtained, and/or improve the scalability of the sequencing processes. NGS technologies can facilitate sequencing of small to large numbers of DNA and/or other nucleic acid samples with high depth of analysis, which can allow detection and deciphering of precise target DNA sequences and/or other suitable sequences. Mixtures of different nucleic acids (e.g., different DNA nucleic acids, etc.) can be simultaneously analyzed, which can facilitate analysis of composition of complex mixtures (e.g., DNA and/or other nucleic acids extracted from a complex ecological community including microorganisms, etc.), and/or rare DNA sequence variants from a pool of conserved sequences (e.g., generation of rare mutations in a small number of cells of a large tissue, etc.). However, construction of sequencing libraries for NGS and/or other sequencing approaches can include library preparation processes (e.g., DNA manipulation, amplification, etc.) that can introduce a variety of biases (e.g., towards different targets such as DNA targets, towards ratios between targets, etc.). Additionally, the number of sequenced reads may not necessarily represent a direct proportion of the nucleic acid molecules (e.g., DNA Molecules) present in the library or in the original mix, which can present difficulties in generating absolute quantitative data (e.g., precise numbers or estimations of the composition of the original biological sample analyzed, etc.).

Further, NGS technologies and/or other suitable sequencing technologies can be used for amplicon-associated sequencing (e.g., analysis associated with a single or small number of gene regions, such as for identification of one or more microorganism taxa in a biological sample, etc.) or metagenome-associated sequencing (e.g., analysis associated with a microbial community and/or other suitable ecological communities of a biological sample, such as including a whole community of DNA as opposed to analysis of a single gene amplicon, etc.). However, each of amplicon-associated sequencing or metagenome-associated sequencing, individually, includes unique advantages and disadvantages.

### BRIEF DESCRIPTION OF THE FIGURES

FIG. 1 includes a flowchart representation of variations of an embodiment of a method;

FIG. 2 includes a flowchart representation of variations of an embodiment of a method;

FIG. 3 includes a flowchart representation of variations of an embodiment of a method;

FIG. 4 includes a flowchart representation of variations of an embodiment of a method;

FIG. 5 includes a flowchart representation of variations of an embodiment of a method;

FIG. 6 includes a specific example of a comparison of assigned reads for 16S sequencing libraries assembled with classical sequencing primers or with UMI-based primers including 4N UMI regions;

FIG. 7 includes a specific example of a comparison of assigned reads for 16S sequencing libraries assembled with UMI-based primes including 4N UMI regions or 8N UMI regions;

FIG. 8 includes a specific example of an improvement of target amplification with adding tagging facilitation molecules for a PCR process using UMI-based primers including 8N UMI regions;

FIGS. 9A-9B include a specific example of a comparison of total number of UMIs assigned per sample when using 4N UMI regions, 8N UMI regions, and tagging facilitation molecules.

FIGS. 10A-10B include a specific example of a comparison of total numbers of sequencing reads assigned per sample when using 4N UMI regions, 5N UMI regions and tagging facilitation molecules

FIGS. 11A-11B include a specific example of a comparison of percent of unique UMIs assigned per sample when using 4N UMI regions, 8N UMI regions, and tagging facilitation molecules;

FIG. 12 includes a specific example of effects of a linker region for 16S amplification with UMI-based primers including 8N UMI regions.

### DESCRIPTION OF THE EMBODIMENTS

The following description of the embodiments is not intended to limit the invention to these embodiments, but rather to enable any person skilled in the art to make and use.

### 1. Overview

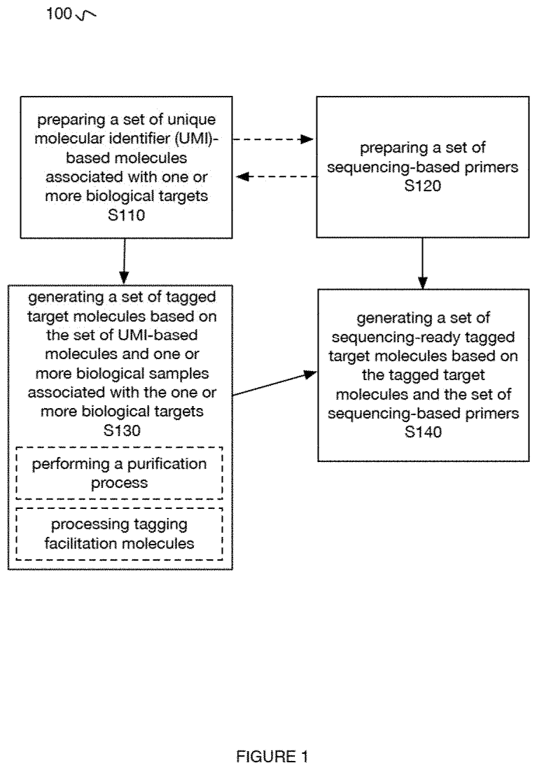

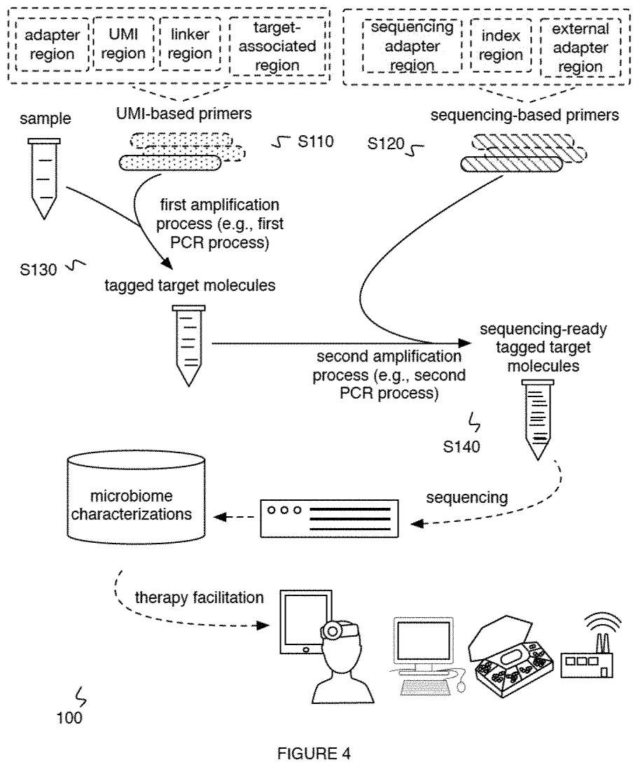

As shown in FIGS. 1 and 4, embodiments of a method 100 for library preparation for sequencing (e.g., next-generation sequencing (NGS), etc.) associated with microorganisms can include: preparing (e.g., determining, generating, etc.) a set of unique molecular identifier (UMI)-based molecules (e.g., UMI-based primers, etc.) associated with one or more targets (e.g., a set of nucleic acid targets; targets associated with microorganisms; etc.) Silo; preparing a set of sequencing-based primers (e.g., adapted for facilitating sequencing, such as next-generation sequencing, associated with microorganisms; etc.) S120; generating a set of tagged target molecules based on the set of UMI-based molecules and one or more biological samples (e.g., at least one biological sample) associated with the one or more targets (e.g., one or more biological samples including nucleic acids associated with the one or more nucleic acid targets; etc.) S130; and/or generating a set of sequencing-ready tagged target molecules (e.g., NGS-ready tagged target molecules; etc.) based on the tagged target molecules and the set of sequencing-based primers S140.

In a specific example, the method 100 (e.g., for NGS associated with microorganisms, etc.) can include preparing a set of UMI-based primers associated with a set of nucleic acid targets (e.g., UMI-based primers including genetic sequences complementary to sequences of one or more nucleic acid targets of the set of nucleic acid targets; etc.) associated with the microorganisms, where each UMI-based primer of the set of UMI-based primers includes a UMI region including a set of random “N” bases, where each N base is selected from any one of an “A” base, a “G” base, a “T” base, and a “C” base; a target-associated region associated with at least one nucleic acid target of the set of nucleic acid targets (e.g., a target-associated region including genetic sequences complementary to sequences of the at least one nucleic acid target; etc.); a linker region (e.g., positioned between the UMI region and the target-associated region; etc.); and/or an adapter region (e.g., including an external adapter region configured to facilitate subsequent processing for preparing sequencing-ready molecules; etc.); preparing a set of sequencing-based primers, where each sequencing-based primer of the set of sequencing-based primers includes an adapter region (e.g., distinct from, similar to, or the same as adapter regions of the UMI-based primers; etc.) associated with the NGS (e.g., an adapter region including sequencing adapter configured to facilitate NGS with one or more NGS technologies, and/or including an external adapter region associated with an external adapter region of the UMI-based primer adapter regions; etc.) and/or including an index region (e.g., a sequencing index region for facilitating combinatorial tagging of different samples; for facilitating multiplexing; etc.); generating a set of tagged target molecules based on a first amplification process (e.g., a first polymerase chain reaction (PCR) process, etc.) with the set of UMI-based primers and at least one biological sample associated with the set of nucleic acid targets; and generating a set of NGS-ready tagged target molecules based on a second amplification process (e.g., a second PCR process, etc.) with the tagged target molecules and the set of sequencing-based primers.

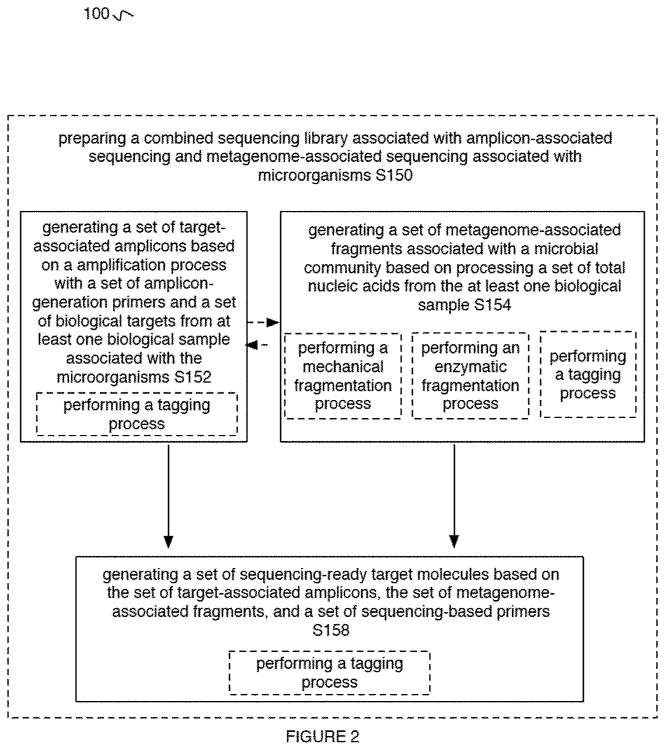

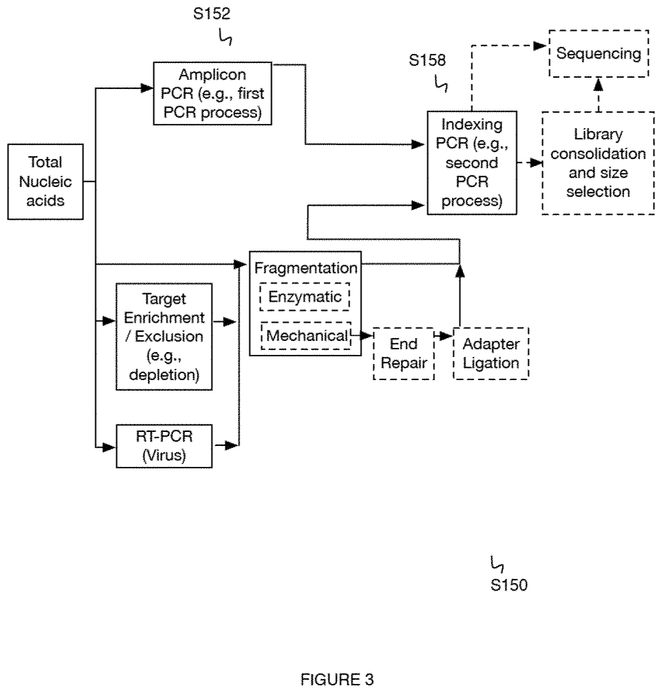

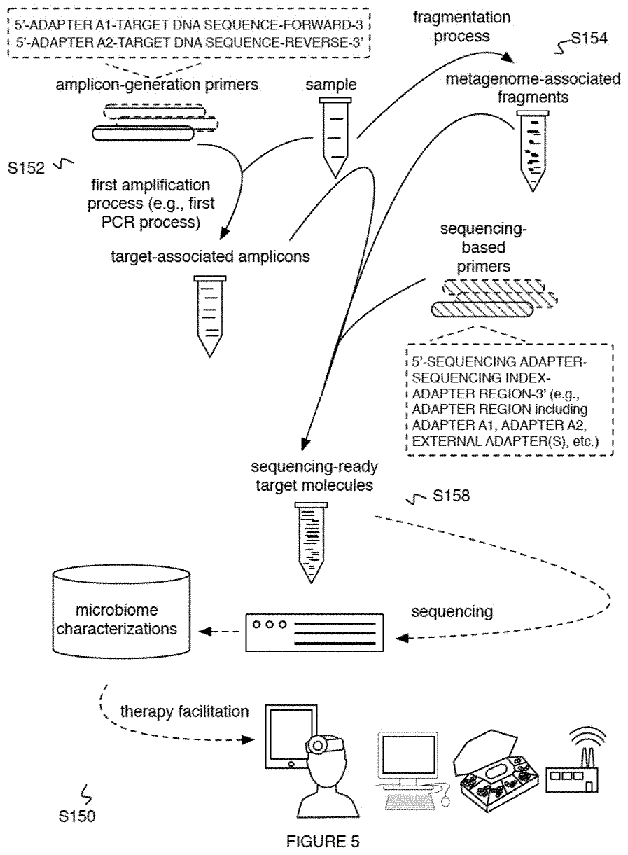

Additionally or alternatively, as shown in FIGS. 2, 3, and 5, embodiments of a method 100 can include preparing a combined sequencing library associated with amplicon-associated sequencing and metagenome-associated sequencing associated with microorganisms S150. In embodiments, the method 100 (e.g., portions of embodiments of the method 100 including preparing a combined sequencing library, etc.) can include generating a set of target-associated amplicons based on a amplification process (e.g., a first PCR process; etc.) with a set of amplicon-generation primers (e.g., UMI-based primers, etc.) and a set of targets from at least one biological sample associated with the microorganisms S152; generating a set of metagenome-associated fragments associated with a microbial community (e.g., corresponding to the microorganisms; etc.) based on processing (e.g., transforming mRNA into cDNA; performing target-capture processes; fragmenting; etc.) a set of total nucleic acids from the at least one biological sample S154; and/or generating a set of sequencing-ready target molecules based on the set of target-associated amplicons, the set of metagenome-associated fragments, and a set of sequencing-based primers (e.g., based on a second amplification process, such as a second PCR process with the target-associated amplicons and/or metagenome-associated fragments; etc.) S158.

Additionally or alternatively, embodiments of the method 100 can include processing (e.g., collecting; sample preparation for facilitating portions of embodiments of the method 100; performing portions of embodiments of the method 100 on; etc.) one or more biological samples from one or more users (e.g., subjects; humans; animals; patients; plants; etc.), such as biological samples collected from one or more collection sites, which can include one or more of a gut site (e.g., as analyzed based on a stool sample, etc.), skin site, nose site, mouth site, genitals site, and/or other suitable physiological sites; determining microbiome characteristics (e.g., microorganism composition characteristics; microorganism function characteristics; characteristics associated with microorganism-related conditions, such as in relation to diagnosis and/or therapy; etc.) based on microorganism sequence datasets (e.g., microorganism sequence datasets generated based on sequencing with sequencing libraries generated from portions of embodiments of the method 100; microorganism sequence datasets generated from bioinformatic analysis associated with sequenced UMI regions, such as UMI regions of sequence-ready tagged target molecules; etc.). However, embodiments of the method 100 can additionally or alternatively include any suitable processes.

Embodiments of the method 100 and/or the system 200 can function to reduce biases associated with sequencing technologies (e.g., biases associated with conventional approaches of DNA library preparation; biases affecting original ratios of individual molecules from one or more original biological samples; biases associated with NGS technologies; etc.), improve quantitative analysis (e.g., analysis of absolute quantities; absolute quantitation of molecules, alleles, gene variants, and/or other components; etc.) of nucleic acids (e.g., DNA molecules; nucleic acids in one or more original samples; etc.) and/or other suitable components (e.g., through normalization of sequencing data based on an assigned number of UMIs for genes of a defined copy number in a sample; etc.); improve processes associated with normalization of RNA transcriptions (e.g., after RNA to DNA conversion; etc.); improve detection of low frequency mutations; improve quantitative single-cell RNA sequencing; improve quantitative analysis of the composition of immune repertoire cells; and/or improve other applications associated with sequencing technologies, such as through improving library preparation for sequencing by improving processing of (e.g., incorporation of; improving efficiency related to incorporation of; improving versatility related to incorporation of; preparation of; determination of; etc.) UMIs (e.g., UMI-based molecules; UMI-regions of UMI-based molecules, etc.) into sequencing libraries (e.g., into target molecules and/or other suitable molecules to be sequenced; etc.). In a specific example, the method 100 can include performing a first and a second PCR process (e.g., a high-efficiency two-step PCR approach; etc.) for tagging (e.g., with UMI regions; etc.) and amplification of target molecules. In a specific example, incorporated UMI regions can facilitate tracking of individual target molecules and/or other suitable molecules (e.g., metagenome-associated fragments; etc.) in complex mixtures (e.g., complex mixtures including a microbial community; etc.), such as through sequencing and/or performing bioinformatics analyses for the UMI regions with NGS technologies, computing systems, and/or other suitable components.

Additionally or alternatively, embodiments of the method 100 and/or system 200 can function to enable preparation of combined sequencing libraries, such as for facilitating performance of (e.g., combination of, etc.) amplicon-associated sequencing and metagenome-associated sequencing simultaneously (e.g., for sequencing with NGS technologies and/or other suitable sequencing technologies; etc.), such as to leverage the advantages (e.g., which can balance out disadvantages; which can facilitate new advantages of reducing analytical biases towards abundant microorganisms of a microbial community, of reducing requirements for degree of characterization of targets such as for primer design including conserved regions for the target and variable regions for differentiation from other taxa, such as in relation to taxonomic markers such as 16S rRNA, rpoB, and/or other markers; etc.) of both amplicon-associated sequencing and metagenome-associated sequencing (e.g., advantages of amplicon-associated sequencing, such as in enabling analysis of a large fraction of organisms in a microbial community that include a target gene and/or other target; advantages of metagenome-associated sequencing, such as in enabling unbiased analyses of microbial communities based on whole community DNA, such as in enabling characterization of a microbial community in relation to both microbiome composition, microbiome function, associated diversity and/or other suitable characteristics; etc.).

In a specific example, the method 100 can include generating combined amplicon (e.g., for taxonomic-related genes such as 16S, 18S, ITS, etc.) and metagenomic DNA libraries (e.g., for enabling metagenomic detection of function-associated genes such as antibiotic genes, virulence genes, human genetic markers; for enabling detection of a plurality of RNA organisms such as viruses; for enabling detection of host and microbial transcribed genes, through mRNA, from a biological sample; etc.). In a specific example, the method 100 can include generating a broad target nucleic acid (e.g., DNA) library.

Additionally or alternatively, embodiments of the method 100 and/or system 200 can function to facilitate provision of data (e.g., microorganism sequence data, etc.) for directed taxonomic profiling (and/or other suitable composition-related analysis) of organisms in one or more biological samples, as well as to facilitate provision of data (e.g., microorganism sequence data, etc.) for genetic functional profiling (and/or other suitable function-related analysis) of the organisms (e.g., through metagenome-associated approaches such as metagenome-associated sequencing, etc.), such as in an additional or alternative manner to performing function-related analysis (e.g., determining microbiome functional features, etc.) based on a standard or known genome.

Additionally or alternatively, embodiments of the method 100 and/or system 200 can function to facilitate microorganism-related detection (e.g., taxonomic detection of organisms of a sample as well as the detection of genes present or expressed in the same sample; detection of organisms with conserved taxonomic genes in a directed fashion, and/or unbiasedly detecting other eukaryotes, prokaryotes, viral organisms, and/or other suitable microorganisms with characterized or non-previously characterized DNA in one or more biological samples; detection of new, unknown, and/or unidentified potential nucleic acid targets, such as by complementing enrichment based protocols such as amplification of specific targets or regions like 16S, 18S, ITS, or any other site-directed based technology, with unbiased metagenomic and/or metatranscriptomic sequencing; detection, in an unbiased manner, of known or identified nucleic acid targets such as associated with antibiotic resistance, virulence factors molecular markers, and other suitable targets of interest, such as by complementing enrichment based protocols; etc.). However, embodiments of the method 100 and/or system 200 can include any suitable functionality.

Embodiments of the method 100 and/or system 200 preferably facilitates library preparation associated with NGS (e.g., NGS technologies). NGS can include any one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), amplicon-associated sequencing (e.g., targeted amplicon sequencing), metagenome-associated sequencing (e.g., metatranscriptomic sequencing, metagenomic sequencing, etc.), sequencing-by-synthesis, tunnelling currents sequencing, sequencing by hybridization, mass spectrometry sequencing, microscopy-based techniques, and/or any suitable NGS technologies.

Additionally or alternatively, embodiments of the method 100 and/or system 200 can facilitate library preparation and/or other suitable processes associated with any suitable sequencing (e.g., any suitable sequencing technologies, etc.), which can include any one or more of: capillary sequencing, Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

Embodiments of the method 100 and/or system 200 can improve sequencing library preparation for facilitating (e.g., based on microorganism sequence datasets derived from sequencing of the sequencing libraries; etc.) characterizations and/or therapies for one or more microorganism-related conditions, which can include one or more of: diseases, symptoms, causes (e.g., triggers, etc.), disorders, associated risk (e.g., propensity scores, etc.), associated severity, behaviors (e.g., caffeine consumption, habits, diets, etc.), and/or any other suitable aspects associated with microorganism-related conditions. Microorganism-related conditions can include one or more disease-related conditions, which can include any one or more of: gastrointestinal-related conditions (e.g., irritable bowel syndrome, inflammatory bowel disease, ulcerative colitis, celiac disease, Crohn's disease, bloating, hemorrhoidal disease, constipation, reflux, bloody stool, diarrhea, etc.); allergy-related conditions (e.g., allergies and/or intolerance associated with wheat, gluten, dairy, soy, peanut, shellfish, tree nut, egg, etc.); skin-related conditions (e.g., acne, dermatomyositis, eczema, rosacea, dry skin, psoriasis, dandruff, photosensitivity, etc.); locomotor-related conditions (e.g., gout, rheumatoid arthritis, osteoarthritis, reactive arthritis, multiple sclerosis, Parkinson's disease, etc.); cancer-related conditions (e.g., lymphoma; leukemia; blastoma; germ cell tumor; carcinoma; sarcoma; breast cancer; prostate cancer; basal cell cancer; skin cancer; colon cancer; lung cancer; cancer conditions associated with any suitable physiological region; etc.), cardiovascular-related conditions (e.g., coronary heart disease, inflammatory heart disease, valvular heart disease, obesity, stroke, etc.), anemia conditions (e.g., thalassemia; sickle cell; pernicious; fanconi; haemolyitic; aplastic; iron deficiency; etc.), neurological-related conditions (e.g., ADHD, ADD, anxiety, Asperger's syndrome, autism, chronic fatigue syndrome, depression, etc.), autoimmune-related conditions (e.g., Sprue, AIDS, Sjogren's, Lupus, etc.), endocrine-related conditions (e.g., obesity, Graves' disease, Hashimoto's thyroiditis, metabolic disease, Type I diabetes, Type II diabetes, etc.), Lyme disease conditions, communication-related conditions, sleep-related conditions, metabolic-related conditions, weight-related conditions, pain-related conditions, genetic-related conditions, chronic disease, and/or any other suitable type of disease-related conditions. Additionally or alternatively, microorganism-related conditions can include one or more human behavior conditions which can include any one or more of: caffeine consumption, alcohol consumption, other food item consumption, dietary supplement consumption, probiotic-related behaviors (e.g., consumption, avoidance, etc.), other dietary behaviors, habitué behaviors (e.g., smoking; exercise conditions such as low, moderate, and/or extreme exercise conditions; etc.), menopause, other biological processes, social behavior, other behaviors, and/or any other suitable human behavior conditions. Conditions can be associated with any suitable phenotypes (e.g., phenotypes measurable for a human, animal, plant, fungi body, etc.).

Embodiments of the method 100 and/or system 200 can be implemented for one or more biological samples from a single user, such as in relation to performing portions of embodiments of the method 100 for preparing a sequencing library from the one or more biological samples from the single user. Additionally or alternatively, embodiments can be implemented for biological samples from a set of users (e.g., population of subjects including the user, excluding the user, etc.), where the set of users can include subjects similar to and/or dissimilar to any other subjects for any suitable type of characteristics (e.g., in relation to microorganism-related conditions, demographic features behavior, microbiome composition and/or function, etc.); implemented for a subgroup of users (e.g., sharing characteristics, such as characteristics affecting portions of embodiments of the method 100; etc.); implemented for plants, animals, microorganisms (e.g., from environmental microbial communities; etc.), and/or any other suitable entities. Thus, information derived from a set of users (e.g., population of subjects, set of subjects, subgroup of users, etc.) can be used to provide additional insight for subsequent users (e.g., in relation to experimental parameters used in performing portions of embodiments of the method 100, etc.). In a variation, an aggregate set of biological samples can be associated with and processed for a wide variety of users, such as including users of one or more of: different demographics (e.g., genders, ages, marital statuses, ethnicities, nationalities, socioeconomic statuses, sexual orientations, etc.), different microorganism-related conditions (e.g., health and disease states; different genetic dispositions; etc.), different living situations (e.g., living alone, living with pets, living with a significant other, living with children, etc.), different dietary habits (e.g., omnivorous, vegetarian, vegan, sugar consumption, acid consumption, caffeine consumption, etc.), different behavioral tendencies (e.g., levels of physical activity, drug use, alcohol use, etc.), different levels of mobility (e.g., related to distance traveled within a given time period), and/or any other suitable characteristic (e.g., characteristics influencing, correlated with, and/or otherwise associated with microbiome composition and/or function, etc.), such as for comparing, for different types of users, amplicon-associated characteristics and metagenome-associated characteristics (e.g., where the amplicon-associated characteristics and metagenome-associated characteristics can be determined based on microorganism sequence datasets derived from combined sequencing libraries for simultaneous amplicon-associated sequencing and metagenome-associated sequencing, etc.). In examples, as the number of users increases, the predictive power of processes implemented in portions of embodiments of the method 100 can increase, such as in relation to characterizing a variety of users based upon their microbiomes (e.g., in relation to different collection sites for samples for the users, etc.). However, portions of embodiments of the method 100 and/or system 200 can be performed and/or configured in any suitable manner for any suitable entity or entities.

Data described herein (e.g., data associated with amplification processes such as PCR processes; data associated with UMI-associated tagging; data associated with sequencing, such as sequencing reads, microorganism sequence datasets, and/or other suitable sequencing data; microbiome features; user data; supplementary data; data associated with microorganism-related conditions; etc.) can be associated with any suitable temporal indicators (e.g., seconds, minutes, hours, days, weeks, etc.) including one or more: temporal indicators indicating when the data was collected (e.g., temporal indicators indicating when a sample was collected; etc.), determined (e.g., temporal indicators indicating when sample processing operations were started, completed, etc.), transmitted, received, and/or otherwise processed; temporal indicators providing context to content described by the data; changes in temporal indicators (e.g., changes in outputs of sample processing operations over time, such as changes in products over cycles of PCR; etc.); and/or any other suitable indicators related to time. Molecules and/or any suitable biological components described herein can include any suitable size (e.g., sequence length, etc.).

Additionally or alternatively, parameters, metrics, inputs, outputs, and/or other suitable data can be associated with value types including any one or more of: scores, individual values, aggregate values, binary values, relative values, classifications, confidence levels, identifiers, values along a spectrum, and/or any other suitable types of values. Any suitable types of data, components (e.g., biological components), products (e.g., of sample processing operations, etc.), described herein can be used as inputs (e.g., for different sample processing operations; models; mixtures; sequencing technologies; etc.), generated as outputs (e.g., of different models; modules; products of sample processing operations; etc.), and/or manipulated in any suitable manner for any suitable components associated with the method 100 and/or system 200.

One or more instances and/or portions of embodiments of the method 100 and/or processes described herein can be performed asynchronously (e.g., sequentially), concurrently (e.g., multiplexing; processing a plurality of samples in portions of embodiments of the method 100; parallel data processing associated with sequencing analysis and/or portions of embodiments of the method 100; etc.), in temporal relation (e.g., substantially concurrently with, in response to, serially, prior to, subsequent to, etc.) to a trigger event (e.g., performance of a portion of an embodiment of the method 100), and/or in any other suitable order at any suitable time and frequency by and/or using one or more instances of the system 200, components, and/or entities described herein.

Additionally or alternatively, portions of embodiments of the method 100 and/or system 200 can facilitate (e.g., where outputs of portions of embodiments of the method 100 and/or system 200 can be subsequently used as inputs; etc.), improve, be used in conjunction with (e.g., serially, in parallel with, etc.), use (e.g., as inputs for portions of embodiments of the method 100 and/or system 200; etc.), have any suitable temporal relationship with, augment, modify, include, and/or can otherwise be associated with that described in U.S. application Ser. No. 15/240,919 filed 18 Aug. 2016, U.S. application Ser. No. 15/649,497 filed 13 Jul. 2017, U.S. Provisional App. No. 62/582,191 filed 6 Nov. 2017, U.S. application Ser. No. 15/811,544 filed 13 Nov. 2017, and U.S. application Ser. No. 15/707,907 filed 18 Sep. 2018, which are each incorporated in their entireties by this reference.

However, the method 100 and/or system 200 can be configured in any suitable manner.

### 2.1 Preparing UMI-Based Molecules

Embodiments of the method 100 can include preparing (e.g., determining, generating, etc.) a set of UMI-based molecules (e.g., UMI-based primers, etc.) associated with one or more targets (e.g., a set of nucleic acid targets; targets associated with microorganisms; etc.) Silo, which can function to prepare molecules used for facilitating tagging (e.g., with UMI-based molecules; UMI regions; adapter regions; linker regions; index regions; etc.) of, amplification of, and/or other suitable processing of one or more targets.

Targets (e.g., targets of interest; known or identified targets; unknown or previously unidentified targets; etc.) can include any one or more of biomarkers; genes (e.g., gene expression markers, etc.); sequence regions (e.g., genetic sequences; sequences identifying a gene, chromosome, microorganism-related condition, conserved sequences, mutations, polymorphisms; amino acid sequences; nucleotide sequences; etc.); nucleic acids (e.g., genomic DNA, chromosomal DNA, extrachromosomal DNA, mitochondrial DNA, plastid DNA, plasmid DNA, cosmid DNA, phagemid DNA, synthetic DNA, cDNA obtained from RNA, single and double stranded DNA, etc.) cells; small molecules; proteins; peptides; targets associated with one or more microorganism-related conditions (e.g., targets informative of diagnosis, prognosis, prediction, and/or therapy associated with one or more microorganism-related conditions; etc.); targets associated with microorganism composition (e.g., targets indicative of taxonomic classification of microorganisms present in a sample; markers indicating presence, abundance, and/or absence of microorganisms of any suitable taxa; etc.) and/or microorganism function (e.g., targets indicative of functional features associated with microorganisms; etc.); lipids; total nucleic acids; whole microorganisms; metabolites; carbohydrates; and/or any suitable types of targets. Portions of embodiments of the method 100 can facilitate library preparation with targets to facilitate improved sequencing (e.g., NGS) and/or analysis of any suitable targets (e.g., through use of UMIs, etc.).

UMI-based molecules are preferably associated with (e.g., including a target-associated region including one or more sequence regions complementary to one or more sequence regions of the one or more targets (e.g., nucleic acid targets, etc.); targeting; amplifiable with; processable with; able to tag; etc.) one or more targets (e.g., microorganism-related nucleic acid targets, etc.), but can additionally or alternatively be associated with any suitable components. UMI-based molecules preferably include UMI-based primers (e.g., for use in one or more amplification processes, such as one or more PCR processes, etc.), but can additionally or alternatively include any suitable types of UMI-based molecules for any suitable purpose.

UMI-based molecules (and/or other suitable molecules, such as primers and/or other molecules described herein) preferably include one or more UMI regions (e.g., where a UMI-based molecule can include a single UMI region; where a UMI-based molecule can include a plurality of UMI regions; etc.). In an example, a UMI region can include a UMI region including a set of random “N” bases (e.g., N deoxynucleotide bases), where each random “N” base is selected from any one of an “A” base, a “G” base, a “T” base, and a “C” base. “N” bases can be continuous (e.g., a strong of “N” bases, etc.), separated (e.g., by defined bases; by any suitable sequence regions; etc.), and/or be located at any suitable sequence position of the UMI-based molecule. UMI regions can include any suitable sequence length (e.g., at least 2 “N” bases; fewer than 21 “N” bases; any suitable number of “N” bases; etc.). UMI region sequence length can be determined based on an amount and/or type of targets to be processes (e.g., quantified, differentiated, etc.), such as where a longer UMI region can facilitate a larger number of random base combinations and a larger set of unique identifiers (e.g., to be used for analyzing a larger number of types of targets to be differentiated; to be used for analyzing samples including a large number of templates and/or gene variants; etc.). In an example, the UMI region can include a 4N UMI region (e.g., a UMI region including 4 “N” bases, etc.). In a specific example, the UMI region can include an 8N UMI region, such as for an amplification process of a 16S gene, such as with an addition of one or more tagging facilitation molecules such as one or more of MgCl₂, dimethyl sulfoxide (DMSO), a thermostable nucleic acid binding protein (e.g., extreme thermostable single-stranded DNA binding protein, etc.), and/or other suitable components. However, UMI regions can be configured in any suitable manner.

UMI-based molecules (and/or other suitable molecules, such as primers and/or other molecules described herein) preferably include one or more target-associated regions. Target-associated regions preferably include sequence regions (e.g., genetic sequences, etc.) but can additionally or alternatively include any suitable types of components (e.g., any suitable components associated with targets, such as bindable to, coupleable to, connectable to, influencing, informing, modifying, and/or with any suitable relationship with targets; etc.). Target-associated regions are preferably associated with (e.g., with sequence complementarity to; targeting; amplifiable with; processable with; etc.) one or more targets (e.g., sequence regions of nucleic acid targets; other suitable components of nucleic acid targets; etc.). In an example, a target-associated region can include a DNA sequence annealable with a complementary target DNA sequence (e.g., of a nucleic acid target). Target-associated regions preferably enable polymerases (e.g., DNA polymerases) to copy and amplify nucleic acid targets and/or other suitable components, but target-associated regions can include any suitable functionality. Target-associated regions can include any suitable length (e.g., at least 15 bases in length; any suitable number of bases; etc.). Alternatively, UMI-based molecules can exclude target-associated regions. However, target-associated regions (and/or other suitable molecules) can be configured in any suitable manner

UMI-based molecules (and/or other suitable molecules, such as primers and/or other molecules described herein) can include one or more linker regions.

Linker regions preferably are without full complementarity (e.g., no complementarity, partial complementarity, etc.) to one or more nucleic acid targets (e.g., nucleic acid targets associated with the target-associated region; etc.). Linker regions can include any suitable length (e.g., where the linker region includes a length fewer than 21 bases, such as for each UMI-based primer of a set of UMI-based primers; a length of any suitable number of bases; etc.). Linker regions are preferably positioned between a UMI region and a target-associated region (e.g., separating a UMI sequence region and a target-associated sequence region; etc.), but can be located at any suitable positions (e.g., any suitable sequence positions; etc.), such as where, for each UMI-based molecule (e.g., for each UMI-based primer of a set of UMI-based primers; etc.), the linker region is positioned between the UMI region and the target-associated region of the UMI-based molecule. In a specific example, UMI-based molecules can include a linker region including a length of seven bases positioned between a target-associated region (e.g., an annealing region) and a UMI region, where the UMI-based molecules can be used in amplifying a segment of 16S from the E. coli genome, where the presence of the linker region can improve efficiency of 16S amplification (e.g., where amplification of the 16S region is smaller when using UMI-based primers including an 8N UMI region and excluding a linker region; etc.). Alternatively, UMI-based molecules (and/or other suitable molecules) can exclude linker regions. However, linker regions can be configured in any suitable manner.

UMI-based molecules (and/or other suitable molecules, such as primers and/or other molecules described herein) can include one or more adapter regions. Adapter regions preferably include external adapter regions (e.g., where an adapter region can include one or more external adapter regions; etc.), which preferably include sequence regions (e.g., sequences, etc.) configured to facilitate sequencing library preparation (e.g., configured to facilitate construction and sequencing of NGS libraries; etc.), but external adapter regions can additionally or alternatively include any suitable components for facilitating sequencing. External adapter regions can include any suitable length (e.g., sequence length; any suitable number of bases; etc.) and/or any suitable sequence regions (e.g., any suitable combination of bases, etc.), which can be determined based on the type of sequencing (e.g., type of sequencing technology used; etc.). Alternatively, UMI-based molecules (and/or other suitable molecules) can exclude adapter regions. However, adapter regions can be configured in any suitable manner.

In a specific example, UMI-based molecules (e.g., UMI-based primers) can include a configuration including “5′-EXTERNAL ADAPTER-UNIQUE MOLECULAR IDENTIFIER-LINKER-TARGET DNA SEQUENCE-3′”, but UMI-based molecules can include any suitable configuration.

UMI-based molecules can include any suitable size (e.g., any suitable sequence length, etc.), and any suitable number and/or types of UMI-based molecules can be prepared and/or used in portions of embodiments of the method 100.

Preparing UMI-based molecules can be performed before and/or after any suitable portions of embodiments of the method 100 (e.g., before or after preparing a set of sequencing-based primers; before or during generation of tagged target molecules; after generation of tagged target molecules for iterative generation of tagged target molecules; etc.), and/or at any suitable time and frequency.

However, preparing UMI-based molecules can be performed in any suitable manner.

### 2.2 Preparing Sequencing-Based Primers

Embodiments of the method 100 can include preparing a set of sequencing-based primers S120, which can function to prepare primers used for facilitating generation of sequencing-ready (e.g., NGS-ready) molecules, such as in relation to improving sequencing associated with microorganisms.

Sequencing-based primers (and/or other suitable molecules described herein) preferably include one or more adapter regions. Adapter regions of sequencing-based primers preferably include one or more sequencing adapter regions, which preferably include sequence regions facilitative of NGS (e.g., sequence regions required by one or more NGS technologies for performing sequencing; sequence regions determined based on a type of NGS technology used; facilitate of NGS technologies; etc.), but sequencing adapter regions can be configured in any suitable manner. Additionally or alternatively, any suitable adapter regions can include sequencing adapter regions. Adapter regions of sequencing-based primers preferably include one or more external adapter regions (e.g., same, similar to, different, complementary to external adapter regions of other adapter regions, such as adapter regions of UMI-based molecules, etc.), but any suitable adapter regions can include the one or more external adapter regions. Adapter regions of sequencing-based primers preferably include one or more index regions (e.g., sequencing index region; etc.), which are preferably configured to facilitate multiplexing, combinatorial tagging of different samples (and/or components of samples, components to be sequences), and/or other suitable functionality associated with NGS and/or other sequencing. An index region preferably includes a defined barcode sequences (e.g., including a length of at least 2 bases and fewer than ii bases; including a length of any suitable number of bases; etc.), but can additionally or alternatively include any suitable components including any suitable length. In a specific example, sequencing-based primers can include a configuration including “5′-SEQUENCING ADAPTER-SEQUENCING INDEX-EXTERNAL ADAPTER-3′”. Adapter regions can include sequencing adapter regions separated from, contiguous with, and/or otherwise positioned relative external adapter regions, but any suitable regions can include any suitable positions relative other regions, and/or any suitable positions. Additionally or alternatively, sequencing-based primers can include any suitable regions (e.g., described herein in relation to primers, etc.) and/or other suitable components. However, sequencing-based primers can be configured in any suitable manner.

Preparing sequencing-based primers can be performed before and/or after any suitable portions of embodiments of the method 100 (e.g., before or after preparing a set of UMI-based molecules, before or after generating tagged target molecules, etc.), and/or at any suitable time and frequency. However, preparing a set of sequencing-based primers can be performed in any suitable manner.

### 2.3 Generating Tagged Target Molecules

Embodiments of the method 100 can include generating a set of tagged target molecules based on a set of UMI-based molecules and one or more biological samples associated with the one or more targets (e.g., biological samples including the one or more targets; biological samples with absence of the one or more targets; etc.) S130, which can function to obtain tagged targets for facilitating downstream sample processing and/or bioinformatics analyses for determining microorganism-related characterizations.

Tagged target molecules preferably include targets (e.g., components including targets, such as total nucleic acids and/or nucleic acid fragments including target sequence regions, etc.) tagged with (e.g., attached with; connected to; coupled with; etc.) one or more UMI-based molecules, but can additionally or alternatively include any suitable components associated with one or more targets and tagged with any suitable molecules. Generating the set of tagged target molecules is preferably based on (e.g., use; process with; perform amplification processes with; etc.) a set of UMI-based molecules (e.g., UMI-based primers, etc.) and one or more biological samples (e.g., tagging components of the one or more biological samples with the set of UMI-based molecules and/or components of the set of UMI-based molecules; etc.), but can additionally or alternatively be based on any suitable components.

Generating the set of tagged target molecules is preferably based on (e.g., includes; uses outputs from; etc.) one or more amplification processes. Amplification processes (e.g., associated with generating the set of tagged target molecules; associated with any suitable portions of embodiments of the method 100; etc.) preferably include one or more PCR processes (e.g., solid-phase PCR, RT-PCR, qPCR, multiplex PCR, touchdown PCR, nanoPCR, nested PCR, hot start PCR, etc.), but can additionally or alternatively include one or more of helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), and/or any other suitable amplification processes. In a specific example, performing a PCR process can include amplifying one or more target DNA sequences by PCR in a thermal cycler using a set of UMI-based primers (e.g., with concentration including or between 20 to 2000 nM; with any suitable concentration; etc.), such as with using DNA polymerase (e.g., including or between 0.02-0.08 units/uL of DNA polymerase; with any suitable concentration; etc.). In a specific example, performing the PCR process can include performing in between and/or including two or three cycles of PCR (e.g., to generate a single copy of each target molecule flanked by a UMI region and an external adapter region; performing the PCR process with one or more tagging facilitation molecules; etc.). However, performing any suitable PCR processes and/or other amplification processes (e.g., in relation to generating the set of tagged target molecules; in relation to any suitable portions of embodiments of the method 100; etc.) can be performed in any suitable manner.

Generating the set of tagged target molecules can additionally or alternatively be based on (e.g., use; process with; perform amplification processes with; etc.) one or more tagging facilitation molecules (e.g., which can be used to improve efficiency and/or versatility related to tagging, such as incorporation of UMI-based molecules to nucleic acid targets; which can be used to improve amplification processes, such as in relation to efficiency; etc.). Tagging facilitation molecules can include any one or more of MgCl₂, dimethyl sulfoxide (DMSO), thermostable nucleic acid binding proteins, betaine, formamide, tween, triton, NP-40, Tetramethyl ammonium chloride (TMAC), bovine serum albumin (BSA), organic and/or inorganic enhancer elements, compounds, salts, small molecules, biomolecules and/or any other suitable molecules configured to facilitate tagging.

In an example, generating a set of tagged target molecules can include performing a first amplification process with a set of UMI-based primers, at least one biological sample, and a set of tagging facilitation molecules including at least one of MgCl₂, dimethyl sulfoxide (DMSO), and a thermostable nucleic acid binding protein. In a specific example, the thermostable nucleic acid binding protein can include a thermostable single-stranded DNA binding protein, where generating the set of tagged target molecules can include performing a first amplification process with the set of UMI-based proteins, the at least one biological sample, and the set of tagging facilitation molecules including MgCl₂ and the thermostable single-stranded DNA binding protein.

In an example, a thermostable nucleic acid binding protein can include an extreme thermostable single-stranded DNA binding protein (e.g., isolated from a hyperthermophilic microorganism; with capability of remaining active after incubation at a high temperature for a threshold period of time, such as at temperatures observed in amplification processes; etc.).

In a specific example, performing a PCR process can be based on (e.g., use, etc.) a set of tagging facilitation molecules including MgCl₂ and a thermostable nucleic acid binding protein (e.g., extreme thermostable single-stranded DNA binding protein), a set of UMI-based primers including 5N UMI regions, and one or more biological samples, such as where using the set of tagging facilitation molecules can improve incorporation of the UMI-based primers with components of the one or more biological samples. Performing PCR processes can be with (e.g., at, associated with, etc.) a thermal cycler (e.g., a conventional thermal cycler), and/or any other suitable systems for facilitating PCR processes.

Generating tagged target molecules (and/or tagging any suitable molecules) can be performed at any suitable time and frequency (e.g., prior to generating sequencing-ready tagged target molecules; during or after generating sequencing-ready tagged target molecules, such as in an iterative product generation approach, etc.).

In a variation, generating a set of tagged target molecules can include performing one or more fragmentation processes, ligation processes, and/or other suitable processes (e.g., in addition to or alternatively to PCR based processes, etc.) such as to tag the one or more targets such as nucleic acid targets (and/or other suitable components of the one or more biological samples, etc.) with the UMI-based molecules. In an example, generating the set of tagged target molecules can include generating fragments based on at least one of an enzymatic process and mechanical process (e.g., enzymatic and/or mechanical fragmentation, etc.) with one or more biological samples (e.g., to generate fragments including the one or more nucleic acid targets, such as target sequences corresponding to targets of interest; to generate fragments from the one or more biological samples; etc.); and performing a ligation process (e.g., blunt-end ligation with ligase enzyme; etc.) for the UMI-based molecules and the fragments (e.g., ligating the UMI-based molecules to the fragments; etc.), such as prior to amplifying target molecules (e.g., target NDA; for sequencing library construction; etc.). In an example, generating the set of tagged target molecules can include generating nucleic acid fragments from at least one biological sample; and ligating the set of UMI-based molecules to the nucleic acid fragments. In examples, performing the one or more fragmentation processes and/or ligation processes can result in indiscriminately tagging all available molecules (e.g., in the solution), whereas, in examples, generating the set of tagged target molecules with a PCR process (e.g., described herein, etc.) can facilitate specific targeting (e.g., of target DNA sequences) for UMI tagging. Ligation processes used for UMI tagging can use same, similar, or distinct UMI-based molecules (e.g., to tag generated fragments, and/or other molecules; etc.) from types of UMI-based molecules used in PCR processes for generating tagged target molecules performing fragmentation processes. In a specific example, UMI-based molecules including a DNA adapter including UMI regions (e.g., including the configuration including “EXTERNAL ADAPTER-UNIQUE MOLECULAR IDENTIFIER-LINKER-TARGET DNA SEQUENCE”, etc.) can be ligated. Additionally or alternatively, additional components (e.g., regions, etc.) can be added before, during, and/or after ligation processes (e.g., adding, such as through PCR processes, additional regions such as by use of primers including the configuration including “5′-SEQUENCING ADAPTER-SEQUENCING INDEX-EXTERNAL ADAPTER-3′”, etc.). However, performing one or more fragmentation processes and/or ligation processes can be performed in any suitable manner.

In a variation, generating the set of tagged target molecules can include a combination (e.g., serial combination; parallel combination; etc.) of at least one PCR process and at least one ligation process. For example, generating the set of tagged target molecules can include performing a PCR process with a set of primers (e.g., including one or more target-associated regions, linker regions, and/or any other suitable components, etc.), such as to increase PCR efficiency and target amplification; and performing a ligation process with one or more UMI-based molecules (e.g., including one or more UMI regions, adapter regions, and/or other suitable components, etc.), such as for adding the UMI-based molecules to products of the PCR process (e.g., amplified nucleic acid targets; etc.). In an example, generating the set of tagged target molecule can include performing a PCR process based on at least one biological sample and a set of primers including a target-associated region associated with at least one target of the set of targets; and ligating a set of UMI-based molecules to products of the PCR process. In a specific example, performing the ligation processes with one or more UMI-based molecules can include performing the one or more ligation processes based on homology and using exonuclease for targeted degradation of one strand of DNA, polymerase, ligase, and/or other suitable components. In a specific example, a UMI-based molecule can include an oligonucleotide including an adapter region (e.g., including an external adapter), a UMI region, a region of any length at the 3′ end which is homologous to the 5′ end of one or more amplicons generated by the at least one PCR processes, and/or any other suitable regions facilitative of ligation processes. However, performing a combination of at least one PCR process and at least one ligation process can be performed in any suitable manner.

Generating the set of tagged target molecules (and/or suitable portions of embodiments of the method 100) can include performing one or more purification processes (e.g., to purify any suitable components; to remove any suitable components; etc.). In an example, generating the set of tagged target molecules can include performing a purification process with products of the first amplification process to remove UMI-based primers of the set of UMI-based primers (and/or to remove other suitable components, etc.) from the products of the first amplification process. In examples, the method 100 can include performing a purification process for products obtained from amplification processes described herein (e.g., a PCR process used to generate a pool of tagged target molecule products, etc.), such as purifying products obtained from a PCR-based amplification process performed with the first set of UMI-based primers. Purification processes can include any one or more of: silica-based DNA binding mini-columns, Solid Phase Reversible Immobilization (SPRI) magnetic beads (e.g., for upscaling and automation, etc.), precipitation of nucleic acids from the biological samples (e.g., using alcohol-based precipitation methods), liquid-liquid based purification techniques (e.g., phenol-chloroform extraction), chromatography-based purification techniques (e.g., column adsorption), purification techniques involving use of binding moiety-bound particles (e.g., magnetic beads, buoyant beads, beads with size distributions, ultrasonically responsive beads, etc.) configured to bind nucleic acids and configured to release nucleic acids in the presence of an elution environment (e.g., having an elution solution, providing a pH shift, providing a temperature shift, etc.), and/or any suitable purification processes. In a specific example, magnetic beads can enable purification of small amounts of products of PCR processes, such as by electrostatic interaction of DNA with the carboxyl coated bead. In a specific example (e.g., as an alternative to, etc.), performing a purification process with magnetic beads can include using between 1:1.2 to 1:0.6 sample to bead volume ratio (e.g., where interaction of small DNA molecules with the beads is disfavored and unspecific products of sizes preferably 100 bp and below are eliminated, etc.). In a specific example (e.g., as an alternative to, etc.), performing a purification process with magnetic beads can include using between 5 to 100 units of Exonuclease I, and/or any other single-strand DNA degrading enzyme, such as to be added to obtained products from any suitable PCR processes, such as to selectively degrade UMI-based molecules (e.g., DNA primers; UMI-based molecules that did not tag molecules of the sample; etc.) and/or other suitable components (e.g., from the first PCR). In a specific example, performing the purification process with magnetic beads can include supplementing the process by adding 1 to 100 units of DpnI restriction enzyme, such as to degrade PCR template DNA. In a specific example, the combination of both enzymatic treatments and/or other suitable processes can be used in addition to or as an alternative to PCR product cleanup approaches. Additionally or alternatively, purification processes can be performed in any suitable manner (e.g., in relation to any suitable portions of embodiments of the method 100, etc.).

However, generating tagged target molecules can be performed in any suitable manner.

### 2.4 Generating Sequencing-Ready Tagged Target Molecules

Embodiments of the method 100 can include generating a set of sequencing-ready tagged target molecules (e.g., NGS-ready tagged target molecules; etc.) based on the set of tagged target molecules and the set of sequencing-based primers S140, which can function to process target molecules (e.g., tagged target molecules) for preparation for sequencing (e.g., NGS, etc.).

Preparing molecules for sequencing preferably includes preparing tagged target molecules for sequencing (e.g., by adding one or more adapter regions and/or more index regions, etc.), but can additionally or alternatively include preparing any suitable molecules for sequencing.

Generating the set of sequencing-ready tagged target molecules is preferably based on (e.g., use; process with; perform amplification processes with; etc.) a set of tagged target molecules and a set of sequencing-based primers (e.g., for incorporation of the sequencing-based primers with the set of tagged target molecules; for adding regions of the sequencing-based primers to the set of tagged target molecules; etc.), but can additionally or alternatively be based on any suitable components. In an example, each UMI-based primer of a set of UMI-based primers (e.g., used in generating the set of tagged target molecules; etc.) can include an external adapter region associated with the NGS; where the set of tagged target molecules (e.g., generated based on the UMI-based primers; etc.) includes the external adapter regions; and where generating a set of sequencing-ready tagged target molecules (e.g., NGS-ready tagged target molecules; etc.) includes annealing a set of sequencing-based primers (e.g., including an adapter region including external adapter regions, such as complementary external adapter regions, etc.) with the tagged target molecules at the external adapter regions of the tagged target molecules. In an example, the method 100 can include generating the set of tagged target molecules based on a first amplification process including a first PCR process; generating a set of sequencing-ready tagged target molecules (e.g., NGS-ready tagged target molecules) based on a second amplification process including a second PCR process with the tagged target molecules and a set of sequencing-based primers; where each sequencing-based primer of the set of sequencing-based primers includes an adapter region (e.g., associated with the sequencing, such as NGS, etc.) and an index region configured to facilitate multiplexing associated with the NGS; and where generating the set of NGS-read tagged target molecules includes adding the index region and the adapter region to tagged target molecules of the set of tagged target molecules, based on the second PCR process with the tagged target molecules and the set of sequencing-based primers. In a specific example, performing a PCR process (e.g., a second PCR process for generating the set of sequencing-ready tagged target molecules) can include using between and/or including 0.02-0.08 units/uL of DNA polymerase, for between and/or including 24-45 cycles of PCR. In a specific example, performing the PCR process (e.g., a second PCR process, etc.) can enable the amplification of clean DNA products from generating the set of tagged target molecules (e.g., products from performing a first PCR process, etc.), which can increase the DNA concentration of nucleic acid targets (e.g., target molecules) to levels suitable for sequencing (e.g., NGS; such as at least 1 pM). In a specific example, generating the set of sequencing-ready tagged target molecules can include adding one or more adapter regions, index regions (e.g., for facilitating multiplexing, etc.), and/or other suitable regions to tagged target molecules and/or other suitable components. In a specific example, generating the set of sequencing-ready tagged target molecule can include adding regions from the set of sequencing-based primers including a configuration including “5′-SEQUENCING ADAPTER-SEQUENCING INDEX-EXTERNAL ADAPTER-3′”.

Generating the set of sequencing-ready tagged target molecules (and/or suitable portions of embodiments the method 100) can additionally or alternatively include performing one or more supplementary amplification processes (e.g., which can function to increase concentrations of tagged target molecules, and/or any other suitable components, etc.). In an example, the method 100 can including performing a supplementary PCR process (e.g., a third PCR process, where generating the tagged target molecules includes performing a first PCR process, and where generating the set of sequencing-ready tagged target molecules includes performing a second PCR process; etc.), such as based on (e.g., using, with, etc.) primers annealing at sequencing adapter regions added by a PCR process (e.g., a second PCR process, etc.) used in generating the set of sequencing-ready tagged targeting molecules. In a specific example, performing a supplementary PCR process can be based on a concentrations (e.g., product concentrations; concentration of products from generating the set of sequencing-ready tagged target molecules; products from a second PCR process; etc.) satisfying a threshold condition (e.g., concentration below 1 pM, etc.).

However, generating the set of sequencing-ready tagged target molecules can be performed in any suitable manner.

### 2.5 Preparing a Combined Sequencing Library

Additionally or alternatively, as shown in FIGS. 2, 3, and 5, embodiments of the method 100 can include preparing a combined sequencing library associated with amplicon-associated sequencing and metagenome-associated sequencing associated with microorganisms S150, which can function to facilitate a combined sequencing technology associated with both amplicon-associated sequencing and metagenome-associated sequencing. In an example, portions of embodiments of the method 100 can include identifying specific microorganisms (and/or performing suitable microbiome characterization in relation to microbiome composition, function, and/or suitable microorganism-related aspects) (e.g., determining abundance of, presence of, absence of, one or more microorganism taxa, etc.) from the microbial community based on a microorganism sequence dataset derived from the set of sequencing-ready target molecules (e.g., derived based on sequencing the set of sequencing-ready target molecules).

Combined sequence libraries preferably include components (e.g., sequencable components, targets, tagged molecules, fragments of total nucleic acids, amplicon-associated components, metagenome-associated components, etc.) associated with amplicon-associated sequencing (e.g., components including amplicons; processed amplicons, such as for preparation for sequencing, such as processed in relation to metagenome-associated components, such as processed in relation to balancing concentration ratios between amplicon-associated components and metagenome-component, such as tagged amplicons; outputs associated with amplicon generation and/or processing; etc.) and metagenome-associated sequencing (components including fragments of total nucleic acids; processed fragments, such as for facilitating sequencing, such as processed in relation to amplicon-associated components; tagged fragments; total nucleic acids themselves; etc.), but can additionally or alternatively include any suitable components.

Amplicons preferably include amplified products from PCR processes (e.g., products including one or more targets such as nucleic acid targets), but can additionally or alternatively include any suitable products associated with amplification processes. Amplicon-associated sequencing preferably includes sequencing associated with analysis of a single or small number of targets (e.g., gene regions), such as for identification of one or more microorganism taxa in a biological sample, but can additionally or alternatively include any suitable sequencing associated with amplicons. Metagenome-associated sequencing preferably includes sequencing associated with analysis of a microbial community and/or other suitable ecological communities (e.g., present in one or more biological samples), such as including a whole community of DNA as opposed to analysis of a single gene amplicon, but can additionally or alternatively include any suitable sequencing associated with microbial communities (e.g., in relation to composition-related analysis; function-related analysis; etc.), ecological communities, groups of microorganisms, and/or metagenome-related aspects.

Portions of preparing a combined sequencing library can be performed with any suitable relationship (e.g., temporal relationship, such as before, after, during, serially, in parallel; relationships regarding components used as inputs and/or generated as outputs; etc.) with portions of embodiments of the method 100.

In variations, portions of preparing one or more combined sequencing library can include any suitable processes (and/or analogous processes) described in relation to tagging target molecules S130, and/or suitable portions of embodiments of the method 100.

However, preparing a combined sequencing library can be performed in any suitable manner.

### 2.5.A Generating Target-Associated Amplicons

Embodiments of the method 100 (e.g., portions of embodiments of the method 100 including preparing a combined sequencing library, etc.) can include generating a set of target-associated amplicons based on an amplification process with a set of amplicon-generation primers and a set of targets (e.g., nucleic acid targets, etc.) from at least one biological sample associated with the microorganisms S152, which can function to generate amplicons facilitative of amplicon-associated sequencing.

Generating the set of target-associated amplicons is preferably based on (e.g., includes; using; processing with; etc.) a PCR process (e.g., a first PCR process of a three-step PCR process for preparing a combined sequencing library in embodiments of the method 100, etc.), such as using a set of amplicon-generation primers, but can additionally or alternatively be based on any suitable amplification processes. Amplicon-generation primers preferably include one or more adapter regions (e.g., adapter regions associated with the target-associated amplicons, such as for facilitating targeting, such as binding of subsequent primers, in subsequent processes of the portions of embodiments of the method 100, such as in facilitating subsequent PCR processes, etc.) and one or more target-associated regions (e.g., for facilitating binding, annealing, and/or other suitable coupling to one or more targets, etc.). In an example, a set of amplicon generation primers can include a first subset of amplicon-generation primers, each amplicon-generation primer of the first subset including a first amplicon-associated adapter region and a first target-associated region associated with a forward sequence of at least one nucleic acid target of a set of nucleic acid targets; and a second subset of amplicon-generation primers, each amplicon-generation primer of the second subset including a second amplicon-associated adapter region and a second target-associated region associated with a reverse sequence of the at least one nucleic acid target of the set of nucleic acid targets, such as where generating the set of target-associated amplicons includes generating the set of target-associated amplicons based on amplification (e.g., a PCR process, etc.) with the first and the second subsets of amplicon-generation primers. In a specific example, a set of amplicon-generation primers can include first primers corresponding to a first primer type and including a configuration including “5′-ADAPTER A1-TARGET DNA SEQUENCE-FORWARD-3′”; and include second primers corresponding to a second primer type and including a configuration including “5′-ADAPTER A2-TARGET DNA SEQUENCE-REVERSE-3′; where “TARGET DNA SEQUENCE” can include any sequencing enabling amplification of one or more nucleic acid targets (e.g., genetic segment of interest, etc.), where “ADAPTER A1” and “ADAPTER A2” can include amplicon-associated adapter regions enable binding of primers and/or other suitable molecules, such as in subsequent portions of embodiments of the method 100 (e.g., subsequent PCR processes; such as in relation to generating a sequencing-ready target molecules; such as in generating sequence-ready molecules; etc.). In a specific example, amplicon-generation primers can include adapter regions including external adaptor regions (e.g., for facilitating annealing with, binding with, and/or other suitable association with sequencing-based primers, such as adapter regions of sequencing-based primers, etc.). However, amplicon-generation primers can include any suitable components and can be configured in any suitable manner.

In a variation, generating the set of target-associated amplicons can include tagging one or more targets (e.g., through amplification processes; etc.), such as tagging one or more targets with one or more UMI-based molecules (e.g., UMI regions and/or other regions of UMI-based molecules, etc.). In an example, the set of amplicon-generation primers can include UMI-based primers (e.g., for use in the corresponding amplification process, etc.). In a specific example, the set of amplicon-generation primers can include a first and second subset of amplicon-generation primers, where the first subset of amplicon-generation primers can include first UMI-based primers, each UMI-based primer of the first UMI-based primers including a first amplicon-associated adapter region, a first target-associated region, and a first UMI region; where the second subset of amplicon-generation primers can include second UMI-based primers, each UMI-based primer of the second UMI-based primers including a second amplicon-associated adapter region, a second target-associated region, and a second UMI region. However, tagging one or more targets (e.g., nucleic acid targets, etc.), and/or performing any suitable processes with UMI-based molecules and/or UMI regions in relation to generating target-associated amplicons, can be performed in any suitable manner.

Amplicons can include any suitable size (e.g., any suitable sequence length, etc.), and can be generated from amplification of any suitable number and/or type of targets and/or other suitable components. However, generating the set of target-associated amplicons can be performed in any suitable manner.

### 2.5.B Generating Metagenome-Associated Fragments

Embodiments of the method 100 (e.g., portions of embodiments of the method 100 including preparing a combined sequencing library, etc.) can include generating a set of metagenome-associated fragments (e.g., metagenome-associated nucleic acid fragments, etc.) associated with a microbial community, based on processing a set of total nucleic acids from one or more biological samples S154, which can function to generate fragments facilitative of metagenome-associated sequencing.

Metagenome-associated fragments can include raw fragments of total nucleic acids (e.g., products of performing fragmentation processes on total nucleic acids of one or more biological samples, etc.), processed fragments of total nucleic acids (e.g., fragments tagged with and/or including one or more adapter regions, UMI-based molecules, any suitable regions, and/or any suitable components; fragments of pre-processed total nucleic acids and/or other suitable components; purified fragments; etc.) and/or any suitable fragments of total nucleic acids and/or other suitable components of one or more biological samples.

Metagenome-associated fragments are preferably associated with one or more microbial communities. A microbial community preferably includes microorganisms (e.g., sharing a common living space, such as a physiological region of a user, such as a sample collection site of a user; etc.) from a plurality of taxa (e.g., taxa including kingdoms, phyla, classes, orders, families, genera, species, subspecies, strains, and/or any other suitable groups of microorganisms; etc.), but can alternatively include only microorganisms from a single taxa. Additionally or alternatively, microbial communities can include interactions between microorganisms, products of interactions between microorganisms, relationships between microorganisms, functional features (e.g., functional profiles, etc.) associated with the microorganisms and/or microbial community, composition features (e.g., taxonomic profiles, etc.) associated with the microorganisms and/or microbial community, and/or any other suitable components and/or features associated with microorganisms and/or microbial communities.

Generating the set of metagenome-associated fragments is preferably based on processing a set of total nucleic acids, but can additionally or alternatively be based on processing any suitable components (e.g., nucleic acid fragments, targets such as nucleic acid targets, other suitable components, etc.).

Generating the set of metagenome-associated fragments (e.g., processing the set of total nucleic acids) preferably includes performing one or more fragmentation processes (e.g., fragmenting; generating fragments of; etc.) with total nucleic acids from the set of total nucleic acids (e.g., all or a subset of the set of total nucleic acids from one or more biological samples, etc.), but can additionally or alternatively include any suitable processes for facilitating metagenome-associated fragment generation. Performing one or more fragmentation processes (e.g., in relation to generating a set of metagenome-associated fragments; in relation to any suitable portions of embodiments of the method 100; etc.) can include any one or more of enzymatic processes (e.g., using transposase-type enzymes for adding defined sequences to one or more ends of cut nucleic acids such as cut DNA, etc.), mechanical processes (e.g., end-repairing obtained DNA fragments of total nucleic acids, and ligating UMI-based molecules and/or other suitable tagging molecules to repaired DNA ends, and/or any suitable types of fragmentation processes. Regions (e.g., sequences) added to outputs of fragmentation processes (e.g., fragments of total nucleic acids) can include adapter regions (e.g., metagenome-associated fragment-generation adapter regions; etc.), such as adapter regions enabling binding of primers and/or other suitable molecules, such as in subsequent portions of embodiments of the method 100 (e.g., subsequent PCR processes; such as in relation to generating sequence-ready target molecules; etc.). However, performing one or more fragmentation processes (e.g., one or more enzymatic processes; one or more mechanical processes; etc.) and/or adding adapter regions and/or other suitable components (e.g., regions, etc.) can be performed in any suitable manner.

In variations, generating a set of metagenome-associated fragments can include tagging one or more fragments (e.g., fragments of total nucleic acids, etc.) and/or other suitable components associated with metagenome-associated fragments and/or total nucleic acids, such as tagging with one or more UMI-based molecules and/or other suitable components (e.g., adapter regions for facilitating subsequent processing with sequencing-based primers, such as adapter regions for sequencing-based primers to anneal to, etc.). In an example, generating a set of metagenome-associated fragments can include generating fragments based on processing a set of total nucleic acids with at least one of an enzymatic process and a mechanical process; and generating the set of metagenome-associated fragments based on ligating UMI-based molecules to the fragments. In an example, generating the set of metagenome-associated fragments can include performing an amplification process (e.g., a PCR process) for adding adapter regions (e.g., external adapter regions, metagenome-associated adapter regions, etc.), UMI regions, and/or any other suitable components to fragments (e.g., of total nucleic acids; etc.). However, tagging the one or more fragments can be performed in any suitable manner (e.g., through amplification processes such as PCR processes; etc.).

Generating the set of metagenome-associated fragments can additionally or alternatively include pre-processing the set of total nucleic acids (e.g., prior to performing one or more fragmentation processes; iteratively with performing fragmentation processes; etc.). Pre-processing (e.g., the set of total nucleic acids; any suitable components), can include any one or more of transforming nucleic acids (e.g., transforming mRNA into cDNA), performing target-capture processes (e.g., enrichment processes, exclusion processes, etc.), perform purification processes, supplemental amplification processes, and/or perform any suitable pre-processing processes. In an example, generating the set of metagenome-associated fragments can include pre-processing the set of total nucleic acids (e.g., prior to fragmentation, etc.), where pre-processing the set of total nucleic acids includes at least one of: transforming mRNA from the set of total nucleic acids into cDNA, performing a first target-capture process to selectively enrich first sequences corresponding to first nucleic acids of the set of total nucleic acids, and performing a second target-capture process to selectively exclude (e.g., deplete, etc.) second sequences corresponding to second nucleic acids of the set of total nucleic acids. Transforming nucleic acids can be used for facilitating detection of expression of target genes and/or other targets (e.g., nucleic acid targets in one or more biological samples), and/or to detect the presence of and/or other suitable characteristics of viruses (e.g., viruses with RNA based genomes, etc.). In an example, pre-processing can include, prior to fragmentation, transforming mRNA in total nucleic acids into cDNA by reverse transcriptase PCR (RT-PCR) (e.g., where RT-PCR can be performed using random primers, such as to reverse transcribe all or substantially all of the mRNA in a sample; or using primers targeting mRNA of interest; etc.) and/or other suitable transformation processes, such as for facilitating fragmentation processes and inclusion in a combined sequencing library. Performing target-capture processes can include enriching or excluding nucleic acids corresponding to target sequences, and/or enriching or excluding (e.g., depleting) suitable types of targets (e.g., prior to fragmentation processes, etc.), such as where target-capture processes can include oligonucleotide-based processes (e.g., using oligonucleotides immobilized or attached to a bead service, where the oligonucleotides can hybridize with sequences in target nucleic acids such as target DNA fragments, etc.). However, pre-processing the set of total nucleic acids can be performed in addition to and/or alternatively to fragmenting total nucleic acids and/or other suitable components, in addition to and/or alternatively to any suitable portion of generating the set of metagenome-associated fragments in embodiments of the method 100, and/or in any suitable manner.

However, generating metagenome-associated fragments can be performed in any suitable manner.

### 2.5.C Generating Sequencing-Ready Target Molecules

Embodiments of the method 100 (e.g., portions of embodiments of the method 100 including preparing a combined sequencing library, etc.) can include generating a set of sequencing-ready (e.g., NGS-ready) target molecules (e.g., associated with one or more targets such as nucleic acid targets, etc.) based on the set of target-associated amplicons, the set of metagenome-associated fragments (e.g., metagenome-associated nucleic acid fragments, etc.), and a set of sequencing-based primers S158, which can function to process one or more target-associated amplicons and/or metagenome-associated fragments, and/or other suitable mixtures (e.g., of amplicon-associated components and metagenome-associated components, etc.) for preparation for sequencing (e.g., NGS; sequencing including simultaneous amplicon-associated sequencing and metagenome-associated sequencing; etc.).

Sequencing-ready target molecules are preferably associated with the one or more targets (e.g., associated with the amplicons) and the microbial community (e.g., associated with the metagenome-associated fragments; where targets include total nucleic acids; where targets are associated with a plurality of taxa of microorganisms; etc.), but can additionally or alternatively be associated with the one or more targets independent of the microbial community; the microbial community independent of the one or more targets; and/or any other suitable targets of interest. Generating sequencing-ready target molecules is preferably based on (e.g., includes) an amplification process (e.g., a second amplification process including a second PCR process, where generating the target-associated amplicons can include a first amplification process including a first PCR process; etc.), with the set of target-associated amplicons, the set of metagenome-associated fragments, and the set of sequencing-based primers. The PCR process preferably includes limited cycles (e.g., fewer than a threshold amount, etc.), but can include any suitable number of cycles, etc.). Performing the amplification process preferably includes adding one or more adapter regions and/or one or more index regions (e.g., through the amplification process) to the components (e.g., the mixture) such as including the target-associated amplicons and/or metagenome-associated fragments, but adapter regions, index regions, and/or other suitable regions can be added in any suitable manner (e.g., ligation processes, etc.). In an example, sequencing-based primers can include index regions (e.g., including sequencing index regions, etc.) configured to facilitate multiplexing associated with the sequencing (e.g., NGS, etc.), and adapter regions associated with the sequencing (e.g., NGS, etc.) and one or more primers and/or adapter regions (e.g., primers used in generating target-associated amplicons such as adapter regions of the primers; adapter regions of target-associated amplicons; adapter regions of metagenome-associated fragments; where sequencing-based primers can include adapter regions complementary, annealable to, and/or otherwise associated with the adapter regions of target-associated amplicons and/or adapter regions of metagenome-associated fragments; and/or other suitable components; etc.). In a specific example, a sequencing-based primer can include a configuration including “5′-SEQUENCING ADAPTER-SEQUENCING INDEX-EXTERNAL ADAPTER-3′”. In a variation, sequencing-based primers can include regions (e.g., adapter regions, etc.) configured to anneal with adapter regions (e.g., amplicon-associated adapter regions; metagenome-associated adapter regions; amplicon-generation adapter regions; metagenome-associated fragment-generation adapter regions; etc.) of target-associated amplicons and/or metagenome-associated fragments, and/or other suitable components (e.g., include in a mixture including the target-associated amplicons and metagenome-associated fragments, etc.). In an example, sequencing-based primers can include regions configured to anneal with amplicon-generation adapter regions and/or other suitable adapter regions (e.g., metagenome-associated adapter regions of metagenome-associated fragments, etc.). Additionally or alternatively, sequencing-based primers associated with S158 can be the same, similar to, or different from sequencing-based primers associated with S140. However, sequencing-based primers can be configured in any suitable manner, and performing amplification processes (e.g., PCR processes) in relation to generating sequencing-ready target molecules can be performed in any suitable manner.

In variations, generating sequencing-ready target molecules can include performing one or more pre-processing processes and/or post-processing processes. In an example, generating sequencing-ready target molecules can include performing a PCR process with the target-associated amplicons, the metagenome-associated fragments, and the set of sequencing-based primers; and cleaning, size-selecting, performing supplementary amplification processes, purifying, enriching, excluding, and/or performing any suitable processes with the products of the PCR process (e.g., for preparing sequencing-ready target molecules suitable for any suitable sequencing technologies; etc.).

In variations, generating the set of sequencing-ready target molecules based on the target-associated amplicons, the metagenome-associated fragments, and/or sequencing-based primers can include any suitable processes (and/or analogous processes) described in relation to generating sequencing-ready tagged target molecules S140 (e.g., based on tagged target molecules and/or sequencing-based primers; etc.). However, generating sequencing-ready target molecules can be performed in any suitable manner.

### 3. Examples

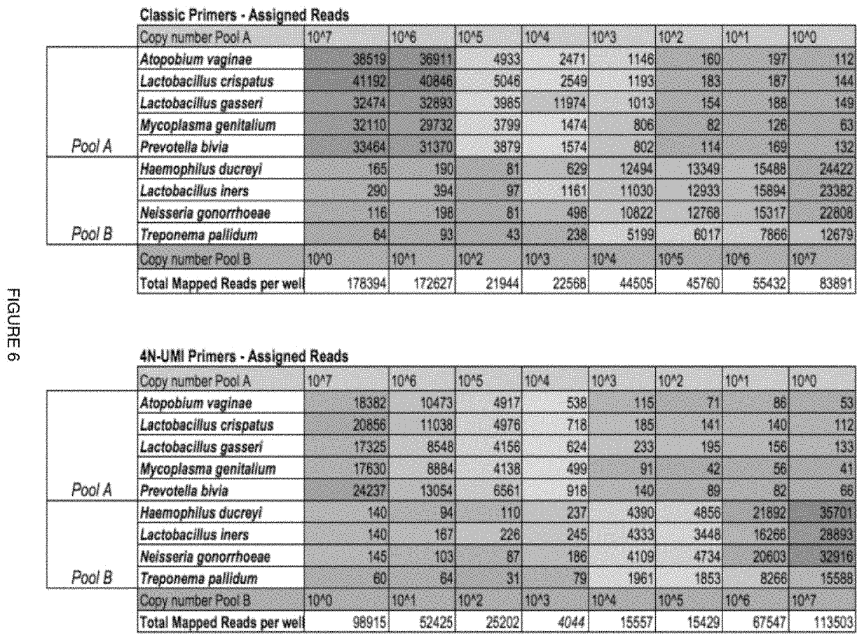

In an example, portions of embodiments of the method 100 can be performed for generating a sequencing library targeting the bacterial 16S ribosomal genes. Generating the sequencing library can include using DNA templates including defined mixes of two bacterial DNA pools, which can be mixed in inverse proportions (e.g., as shown in FIG. 6). In comparing the number of sequencing reads assigned to each member of the pool, it can be shown that comparable number of reads can be obtained for UMI-excluding primers (e.g., primers without a UMI region, etc.) and UMI-based primers in each condition, and for each organism detected.

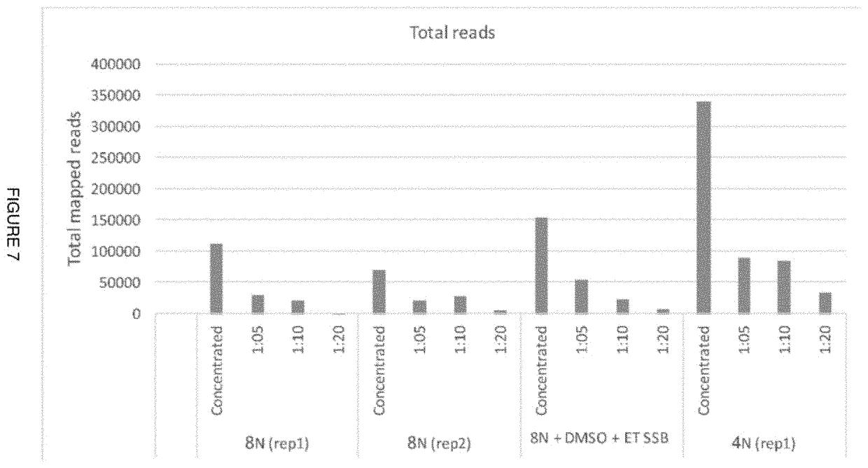

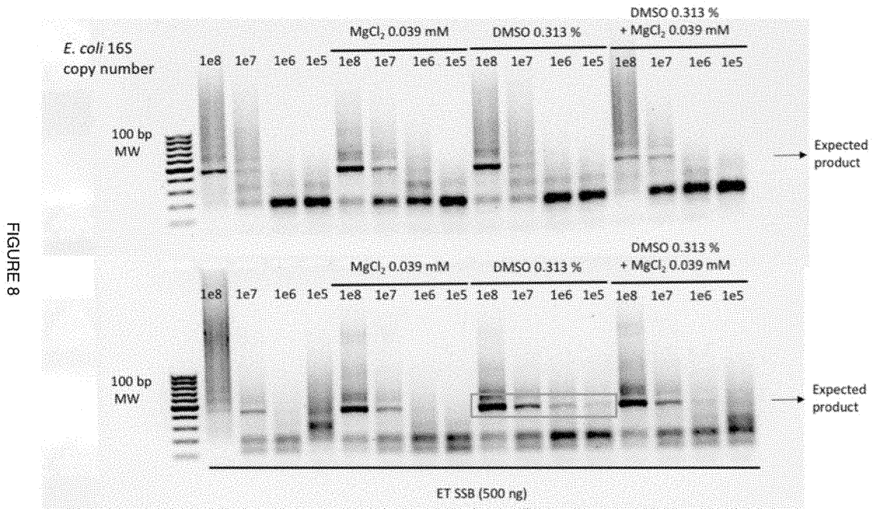

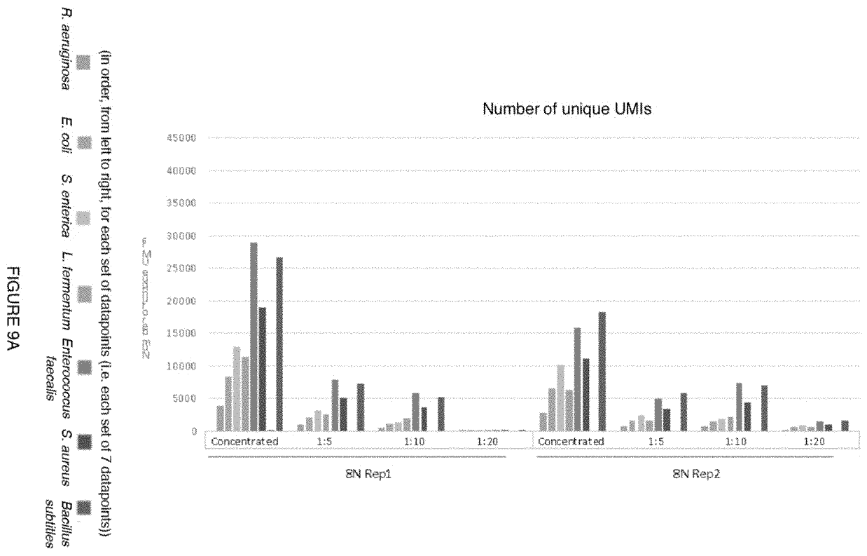

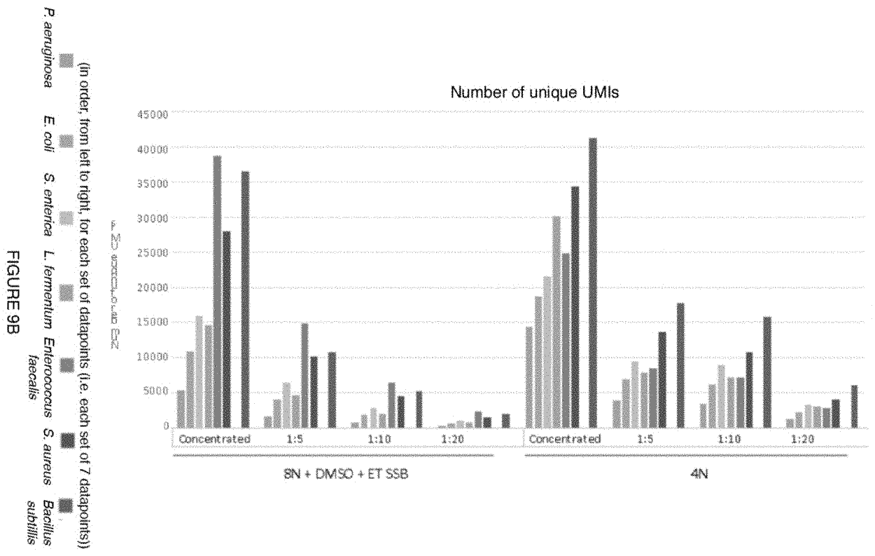

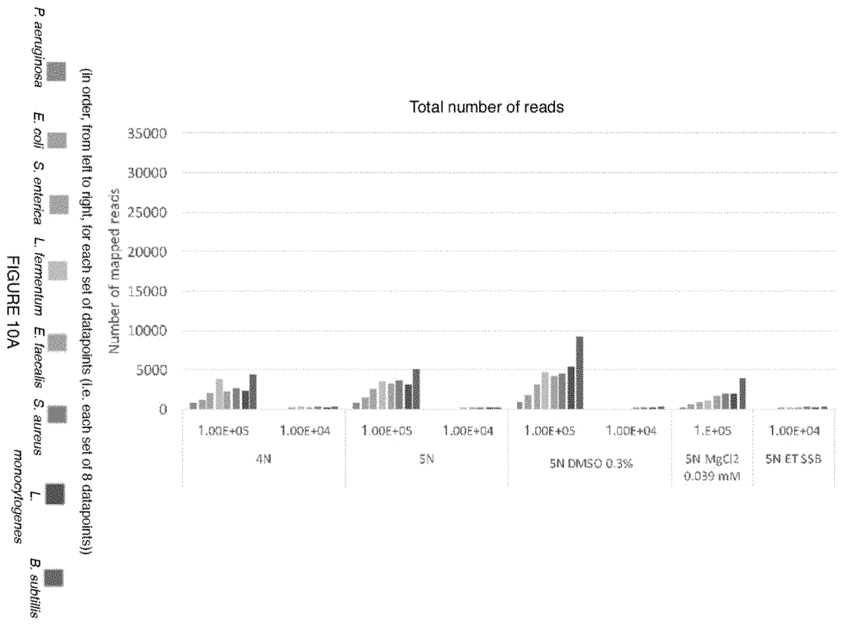

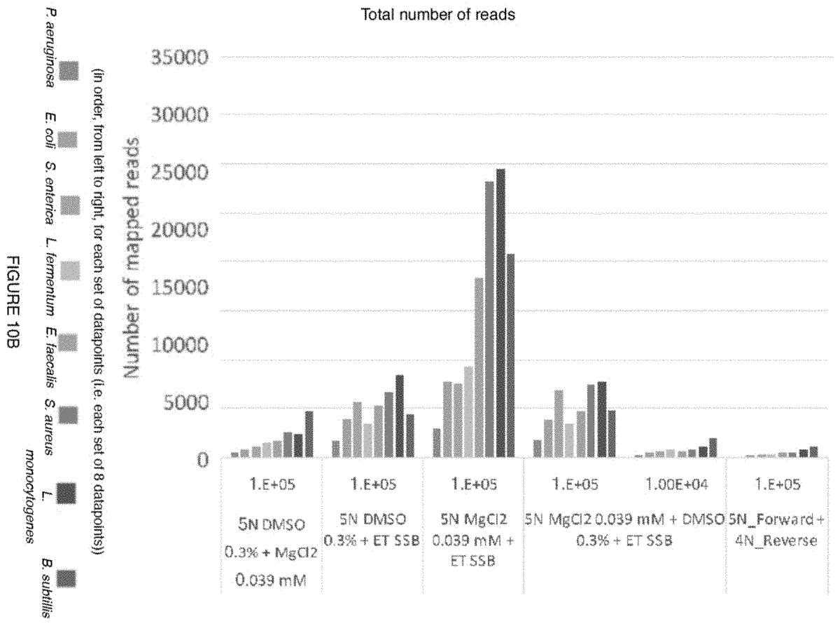

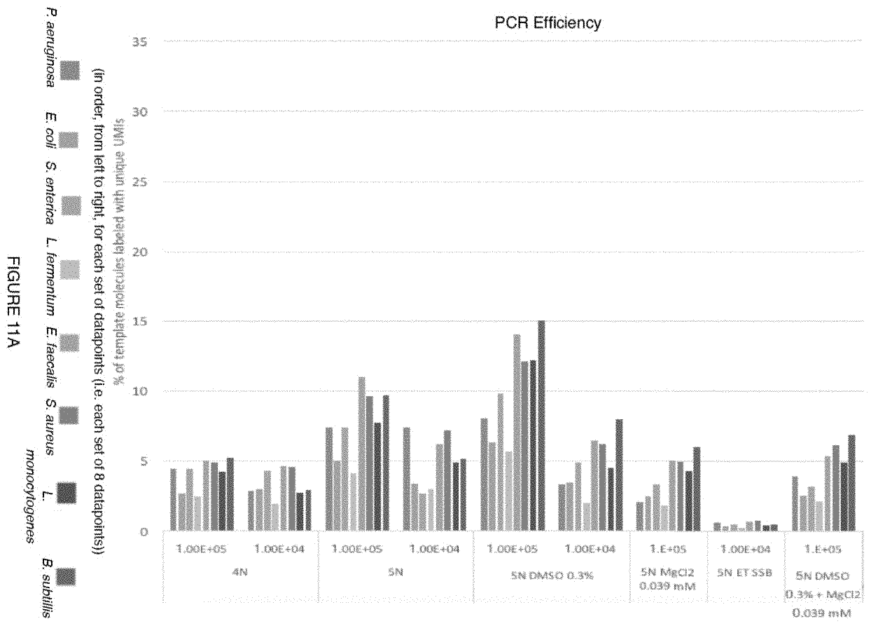

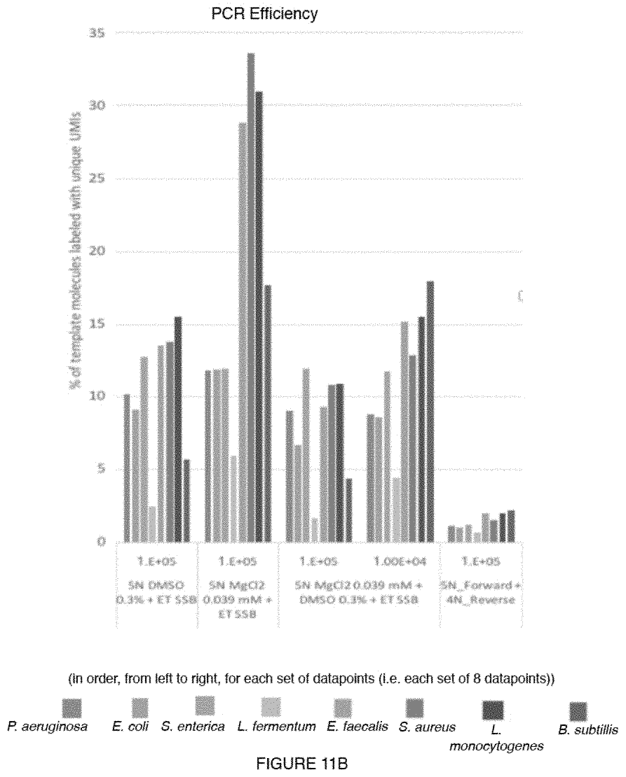

In an example, UMI-based primers including 4N UMI regions or 8N UMI regions can be applied to generate a sequencing library, such as where, for specific applications (e.g., as shown in FIG. 7), a number of assigned sequencing reads can decrease when the number of “N” bases is increased from 4N to 8N (and/or increased in generally), such as where efficiency of tagging can have an inverse correlation with the number of “N” bases. In the example, tagging facilitation molecules can be added for improving tagging efficiency (e.g., efficiency associated with a PCR process of generating tagged target molecules; etc.). In a specific example, as shown in FIG. 8, adding a set of tagging facilitation molecules including MgCl₂, DMSO, and/or an extreme thermostable extreme thermostable single-stranded DNA binding protein to a PCR process using UMI-based primers including 8N UMI regions can improve amplification and/or tagging efficiency (e.g., where, as shown in FIG. 8, amplification of 16S gene using a single DNA template of E. coli genomic DNA can be improved for a range of DNA inputs, as analyzed by agarose gel electrophoresis; etc.). In a specific example, as shown in FIG. 9A-9B, adding tagging facilitation molecules for PCR processes using UMI-based primers including 4N UMI regions or 8N UMI regions can improve tagging efficiency (e.g., higher number of different UMI tags, etc.). In a specific example, as shown in FIG. 10A-10B, adding tagging facilitation molecules for PCR processes using UMI based primers including 4N UMI regions or 5N UMI regions (e.g., as shown in FIG. 10A-10B) can result in an increased number of reads (e.g., for a microbial community standard sample, etc.), such as where 30%, in a specific example (e.g., as shown in FIG. 11A-11B, of target sequences can show unique UMIs. However, adding tagging facilitation molecules can confer any suitable degree of improvement.

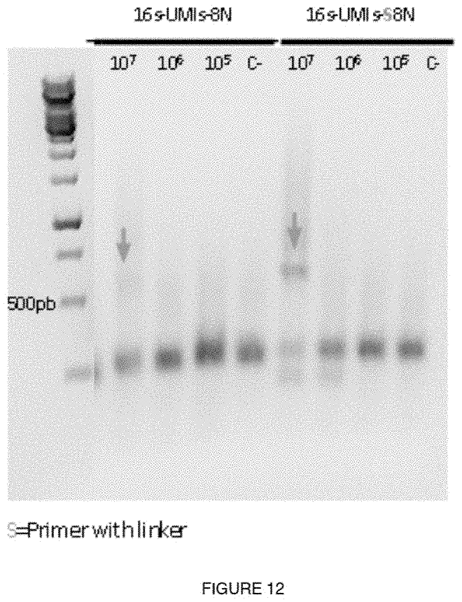

In an example, using UMI-based molecules including one or more linker regions (e.g., separating UMI regions and target-associated regions) can improve efficiency of amplification with primers (e.g., where UMI regions of greater “N” length are used, etc.). In a specific example, as shown in FIG. 12, amplification of a 16S region can be improved through using UMI-based primers including seven base-length linker regions separating UMI regions and target-associated regions.

In an example, portions of embodiments of the method 100 can include preparing combined sequencing libraries from human stool biological samples, but combined sequence libraries can additionally or alternately be prepared from any suitable biological samples (e.g., from any suitable users; from any suitable collection sites; etc.). In specific examples, a combined sequence library can be constructed from a stool sample from a single user; bacterial taxa analysis of samples from a plurality (e.g., hundreds, etc.) of sequencing runs can show statistically significant reproducible diversity (e.g., indicating robustness and consistency; etc.). In a specific example, a combined sequencing library can lead to results illustrating inclusion of all species (and/or other suitable taxa) represented in amplicon-associated components of the combined sequencing libraries, and higher representation of bacterial taxa shown to be underrepresented when using only amplicon-focused approaches (e.g., Tenericutes phylum, etc.). In specific examples, processes associated with preparing a combined sequencing library can be used for identification of different organisms and specific nucleic acid targets of interest, by using amplicon-associated processes to identify the presence or absence of a given microorganism (e.g., including and/or based on 16S regions, 18S regions, ITS, etc.), and by using metagenome-associated processes to identify the nucleic acid target of interest (e.g., antibiotic resistance genes, virulence factors, secretion systems, etc.) and/or other suitable targets

In examples, the method 100 and/or system 200 can confer improvements over conventional approaches. Specific examples of the method 100 and/or system 200 can confer technologically-rooted solutions to at least the challenges associated with conventional approaches. In examples, the technology can transform entities (e.g., biological samples, targets such as nucleic acid targets, primers, UMI-based molecules, users, etc.) into different states or things. In a specific example, nucleic acid targets can be transformed into sequencing-ready target molecules and/or sequencing-ready tagged target molecules, such as adapted for improved sequencing (e.g., associated with reduced biases, improved analyses such as improved quantification, etc.). In a specific example, improved sequencing libraries can be prepared, leading to improved microbiome characterizations, such as for facilitating improved diagnosis and/or therapy associated with one or more microorganism-related conditions, thereby transforming one or more users. However, in examples, the technology can transform entities in any suitable manner.

In examples, the technology can improve technical fields of at least sequencing library preparation, sample processing, genomics, molecular biology, microbiology, diagnostics, therapeutics, digital medicine, modeling, and/or other suitable technical fields. However, in specific examples, the technology can provide any other suitable improvements, such as by performing portions of embodiments of the method 100 and/or system 200.

Embodiments of the method 100 and/or system 200 can include every combination and permutation of the various system components and the various method processes, including any variants (e.g., embodiments, variations, examples, specific examples, figures, etc.), where portions of embodiments of the method 100 and/or processes described herein can be performed asynchronously (e.g., sequentially), concurrently (e.g., in parallel), or in any other suitable order by and/or using one or more instances, elements, components of, and/or other aspects of the system 200 and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method 100 and/or system 200 can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method 100, system 200, and/or variants without departing from the scope defined in the claims.

We claim:

1. A method for library preparation for next generation sequencing (NGS), the method comprising:
preparing a set of unique molecular identifier (UMI)-based primers associated with a set of nucleic acid targets, wherein each UMI-based primer of the set of UMI-based primers comprises:
a UMI region comprising a set of random “N” bases, wherein each random “N” base is selected from any one of an “A” base, a “G” base, a “T” base, and a “C” base; and
a target-associated region associated with at least one nucleic acid target of the set of nucleic acid targets;
preparing a set of sequencing-based primers, wherein each sequencing-based primer of the set of sequencing-based primers comprises an adapter region associated with the NGS;
generating a set of tagged target molecules based on a first amplification process with the set of UMI-based primers and at least one sample associated with the set of nucleic acid targets; and
generating a set of NGS-ready tagged target molecules based on a second amplification process with the tagged target molecules and the set of sequencing-based primers.

2. The method of claim 1, wherein the each UMI-based primer of the set of UMI-based primers further comprises a linker region without full complementarity to the at least one nucleic acid target associated with the target-associated region.

3. The method of claim 2, wherein the linker region comprises a length fewer than 21 bases.

4. The method of claim 2, wherein, for each UMI-based primer of the set of UMI-based primers, the linker region is positioned between the UMI region and the target-associated region.

5. The method of claim 2,
wherein each UMI-based primer of the set of UMI-based primers further comprises an external adapter region associated with the NGS,
wherein the set of tagged target molecules comprises the external adapter regions, and
wherein generating the set of NGS-ready tagged target molecules comprises annealing the set of sequencing-based primers with the tagged target molecules at the external adapter regions of the tagged target molecules.

6. The method of claim 1, wherein generating the set of tagged target molecules comprises performing the first amplification process with the set of UMI-based primers, the at least one biological sample, and a set of tagging facilitation molecules comprising at least one of MgCl₂, dimethyl sulfoxide (DMSO), a thermostable nucleic acid binding protein, betaine, formamide, tween, triton, NP-40, Tetramethyl ammonium chloride (TMAC), and bovine serum albumin (BSA).

7. The method of claim 6, wherein the thermostable nucleic acid binding protein comprises a thermostable single-stranded DNA binding protein, and wherein generating the set of tagged target molecules comprises performing the first amplification process with the set of UMI-based proteins, the at least one sample, and the set of tagging facilitation molecules comprising MgCl₂ and the thermostable single-stranded DNA binding protein.

8. The method of claim 1, wherein generating the set of tagged target molecules comprises performing a purification process with products of the first amplification process to remove UMI-based primers of the set of UMI-based primers from the products of the first amplification process.

9. The method of claim 1,
wherein the first amplification process comprises a first polymerase chain reaction (PCR) process,
wherein the second amplification process comprises a second PCR process,
wherein the each sequencing-based primer of the set of sequencing-based primers further comprises an index region configured to facilitate multiplexing associated with the NGS; and
wherein generating the set of NGS-ready tagged target molecules comprises adding the index region and the adapter region to tagged target molecules of the set of tagged target molecules, based on the second PCR process with the tagged target molecules and the set of sequencing-based primers.

10. A method for library preparation for next generation sequencing (NGS) sequencing, the method comprising:
generating a set of target-associated amplicons based on a first amplification process with a set of amplicon-generation primers and a set of nucleic acid targets from at least one sample;
generating a set of metagenome-associated fragments, based on processing a set of total nucleic acids from the at least one sample;
generating a set of sequencing-ready target molecules based on the set of target-associated amplicons, the set of metagenome-associated fragments, and a set of sequencing-based primers, wherein the set of sequencing-ready target molecules is associated with the set of nucleic acid targets.

11. The method of claim 10, wherein the set of amplicon-generation primers comprises:
a first subset of amplicon-generation primers, each amplicon-generation primer of the first subset comprising a first amplicon-associated adapter region and a first target-associated region associated with a forward sequence of at least one nucleic acid target of the set of nucleic acid targets; and
a second subset of amplicon-generation primers, each amplicon-generation primer of the second subset comprising a second amplicon-associated adapter region and a second target-associated region associated with a reverse sequence of the at least one nucleic acid target of the set of nucleic acid targets,
wherein generating the set of target-associated amplicons comprises generating the set of target-associated amplicons based on amplification with the first and the second subsets of amplicon-generation primers.

12. The method of claim 11,
wherein the first subset of amplicon-generation primers comprises first unique molecular identifier (UMI)-based primers, each UMI-based primer of the first UMI-based primers comprising the first amplicon-associated adapter region, the first target-associated region, and a first UMI region;
wherein the second subset of amplicon-generation primers comprises second UMI-based primers, each UMI-based primer of the second UMI-based primers comprising the second amplicon-associated adapter region, the second target-associated region, and a second UMI region.

13. The method of claim 11, wherein generating the set of metagenome-associated fragments comprises generating the set of metagenome-associated fragments comprising added adapters, based on at least one of a ligation process and an amplification process.

14. The method of claim 13, wherein the set of sequencing-based primers comprises:
metagenome-associated adapter regions associated with the NGS and the added adapters of the set of metagenome-associated fragments.

15. The method of claim 14, wherein each of the set of sequencing-based primers comprises:
index regions configured to facilitate multiplexing associated with the NGS; and
adapter regions associated with the NGS, the set of target-associated amplicons, and the set of metagenome-associated fragments.

16. The method of claim 15, wherein the adapter regions of the set of sequencing-based primers are associated with the NGS, the added adapters of the set of metagenome-associated fragments, the first amplicon-associated adapter regions of the first subset of amplicon-generation primers, and the second amplicon-associated adapter regions of the second subset of amplicon-generation primers.

17. The method of claim 10, wherein generating the set of metagenome-associated fragments comprises:
generating fragments based on processing the set of total nucleic acids with at least one of an enzymatic process and a mechanical process; and
generating the set of metagenome-associated fragments based on ligating unique molecular identifier (UMI)-based molecules to the fragments.

18. The method of claim 10, wherein generating the set of metagenome-associated fragments comprises pre-processing the set of total nucleic acids prior to fragmentation, wherein pre-processing the set of total nucleic acids comprises at least one of:
transforming mRNA from the set of total nucleic acids into cDNA,
performing a first target-capture process to selectively enrich first sequences corresponding to first nucleic acids of the set of total nucleic acids, and
performing a second target-capture process to selectively exclude second sequences corresponding to second nucleic acids of the set of total nucleic acids.

19. The method of claim 10, further comprising identifying specific microorganisms from the microbial community based on a microorganism sequence dataset derived from the set of sequencing-ready target molecules.

20. A method for library preparation for sequencing associated with microorganisms, the method comprising:
preparing a set of unique molecular identifier (UMI)-based molecules associated with a set of nucleic acid targets, wherein each UMI-based molecule of the set of UMI-based molecules comprises a UMI region comprising a set of random “N” bases, wherein each random “N” base is selected from any one of an “A” base, a “G” base, a “T” base, and a “C” base;
preparing a set of sequencing-based primers, wherein each sequencing-based primer of the set of sequencing-based primers is configured to facilitate the sequencing;
generating a set of tagged target molecules based on the set of UMI-based molecules and at least one sample associated with the set of nucleic acid targets; and
generating a set of sequencing-ready tagged target molecules based on an amplification process with the set of tagged target molecules and the set of sequencing-based primers.

21. The method of claim 20, wherein generating the set of tagged target molecules comprises:
performing a polymerase chain reaction (PCR) process based on the at least one sample and a set of primers comprising a target-associated region associated with at least one nucleic acid target of the set of nucleic acid targets; and
ligating the set of UMI-based molecules to products of the PCR process.

22. The method of claim 20, wherein generating the set of tagged target molecules comprises
generating nucleic acid fragments from the at least one sample; and
ligating the set of UMI-based molecules to the nucleic acid fragments.
